(19) United States
(12) Patent Application Publication
(10) Pub. No.: US 2019/0046062 A1
(43) Pub. Date: Feb. 14, 2019

(54) CORONARY SINUS ELECTROPHYSIOLOGY MEASUREMENTS DEVICE AND METHODS

(71) Applicant: The Medical Research, Infratructure and Health Services Fund of the Tel Aviv Medical Center, Tel-Aviv (IL)

(72) Inventors: Lior YANKELSON, Tel-Aviv (IL); Omer SHEZIFI, Haifa (IL); Dima PINHASOV, Kiryat-Yam (IL)

(21) Appl. No.: 16/068,689

(22) PCT Filed: Jan. 5, 2017
(86) PCT No.: PCT/IL2017/050020

§ 371 Date: Jul. 9, 2018

Related U.S. Application Data
(60) Provisional application No. 62/330,365, filed on May 2, 2016.
(60) Provisional application No. 62/100,928, filed on Jan. 8, 2015.

(30) Foreign Application Priority Data
Jan. 7, 2016 (IL) PCT/IL2016/050024

(51) Int. Cl. A61B 5/042; A61B 5/00

(52) U.S. Cl. CPC A61B 5/0422; A61B 18/1492; A61B 5/6859

(57) ABSTRACT

An electrode array configured to be inserted at least partially into a blood vessel including:
a self-expandable array body;
at least two axially spaced-apart electrodes connected to said array body; wherein at least part of said array body expands to an open conformation and pushes at least one selected electrode against a blood vessel inner tissue with a force designed not to damage venous tissue.

### RELATED APPLICATION

This application is a CIP (Continuation In-Part) of PCT Patent Application No. PCT/IL2016/050024 filed on Jan. 7, 2016. In addition, this application claims the benefit of priority under 35 USC § 119(e) of U.S. Provisional Patent Application No. 62/330,365 filed May 2, 2016. The contents of these applications are all incorporated by reference as if fully set forth herein in their entirety.

### FIELD AND BACKGROUND OF THE INVENTION

The present invention, in some embodiments thereof, relates to an electrode array and, more particularly, but not exclusively, to an electrode array configured to be inserted into the coronary sinus.

Electrophysiological (EP) measurements of signals propagating through the heart tissue are often used to diagnose various pathologies of the heart, for example arrhythmia. A typical system for EP measurements comprises an electrode carrying catheter configured to be inserted into a blood vessel, and an EP measuring device connected to the catheter.

U.S. Pat. No. 6,064,905 describes a mapping catheter which comprises a catheter body, a handle and a tip section. The catheter body has an outer wall, proximal and distal ends and at least one lumen extending therethrough (abstract).

### SUMMARY OF THE INVENTION

Following are some examples of some embodiments of the invention:

### Example 1

An electrode array configured to be inserted at least partially into a blood vessel comprising:
a self-expandable array body;
at least two axially spaced-apart electrodes connected to said array body;
wherein at least part of said array body expands to an open conformation and pushes at least one selected electrode against a blood vessel inner tissue with a force designed not to damage venous tissue.

### Example 2

The electrode array of example 1, wherein said array body comprises:
an elongated shaft;
at least three spaced-apart flexible elements, connected with their proximal ends to said elongated shaft in at least three axially spaced-apart locations along said shaft, wherein at least one of said three flexible elements moves to a relaxed state and pushes said blood vessel inner tissue with its distal end when said array body expands;
wherein at least one of said flexible elements is connected to at least one of said electrodes at its distal end, and wherein said array body expands, said flexible element pushes said electrode against said blood vessel inner tissue.

### Example 3

The electrode array of examples 1 or 2, wherein expansion of said array body, anchors said electrode array at least partly within a tubular blood vessel with a diameter-length ratio of at least 1:3.

### Example 4

The electrode array of examples 1 or 2, wherein said electrode is pushed against the blood vessel inner tissue with a force of 2-30 gr when said array body expands.

### Example 5

The electrode array of example 2, wherein said flexible elements make contact with said blood vessel inner tissue with a force of 2-30 gr when said array body expands.

### Example 6

The electrode array of examples 1 or 2, wherein said electrodes contact point with said blood vessel inner tissue allows EP measurements and/or electric field application when said electrode is pushed against the tissue.

### Example 7

The electrode array of examples 1 or 2, wherein said electrodes are pushed against the inner tissue of the coronary sinus with a force that does not cause an injury of the tissue.

### Example 8

The electrode array of example 1, wherein said open conformation of said array body is a helical conformation with a fixed diameter in the range of 5-12 mm.

### Example 9

The electrode array of example 1, wherein said open conformation of said array body is a helical conformation with a varying diameter of 5-12 mm.

### Example 10

The electrode array of example 1, wherein said open conformation of said array body is a helical conformation with a conical shape that has a smaller diameter at its distal section and a wider diameter at its proximal section.

### Example 11

The electrode array of example 10 wherein said smaller diameter is in the range of 5-8 mm, and wherein said wider diameter is in the range of 9-12 mm.

### Example 12

The electrode array of example 2, wherein said relaxed state of said flexible elements forms a similar acute angle between all flexible elements connected to said elongated shaft.

### Example 13

The electrode array of example 2, wherein said flexible elements connected to a distal location of said elongated shaft form a smaller acute angle in a relaxed state compared to flexible elements connected to a more proximal location of said elongated shaft.

### Example 14

The electrode array of example 2, wherein each of said flexible elements found in said relaxed state, form a circle with a diameter of 6-12 mm when said electrode array rotates.

### Example 15

The electrode array of example 2, wherein flexible elements connected to said elongated shaft at proximal locations, form a circle with a diameter of 10-12 mm when they are found in said relaxed state, and wherein flexible elements connected to said elongated shaft at distal locations, form a circle with a diameter of 6-8 mm when they are found in said relaxed state and upon rotation of said electrode array.

### Example 16

The electrode array of examples 1 or 2 further comprising a cylindrical sleeve for covering said electrode array when said electrode array is introduced into said blood vessel and/or for protecting said blood vessel tissue during the insertion of said electrode array into the lumen of said blood vessel.

### Example 17

The electrode array of example 16 wherein movement and/or rotation of said cylindrical sleeve allows expansion or collapse of at least part of said array body.

### Example 18

The electrode array of example 16, wherein movement and/or rotation of said cylindrical sleeve allows deployment and/or collapse of at least one selected electrode.

### Example 19

The electrode array of example 16, wherein movement and/or rotation of said cylindrical sleeve allows deployment and/or collapse of at least one selected flexible element.

### Example 20

The electrode array of examples 1 or 2, wherein pushing of said at least one electrode and/or at least one flexible element allows anchoring of said electrode array within said blood vessel.

### Example 21

The electrode array of examples 1 or 2, wherein the distance between two axially spaced apart electrodes, located on two axially spaced apart flexible elements is at least 10 mm.

### Example 22

The electrode array of example 2, wherein each of said flexible elements is electrically isolated from all the other flexible elements.

### Example 23

The electrode array of example 2, wherein each of said flexible elements is mechanically separated from all the other flexible elements for placing said electrode array within a blood vessel with a varying inner diameter.

### Example 24

The electrode array of examples 1 or 2, wherein each of said electrodes is separately connected via wires to an EP measuring device located outside the body.

### Example 25

The electrode array of example 2, wherein at least one electrode is configured to be pushed by said at least one flexible element to a tissue near the coronary sinus ostium.

### Example 26

The electrode array of example 1, further comprising at least one force sensor located on the array body for measuring the force applied by said array on said blood vessel tissue when said array body expands and/or during the insertion of said electrode array into the lumen of said blood vessel.

### Example 27

The electrode array of example 2, further comprising at least one force sensor located on at least one of said flexible elements and/or on said shaft for measuring the force applied by said at least one flexible element and/or said shaft on said blood vessel tissue when said array body expands.

### Example 28

The electrode array of examples 1 or 2, further comprising at least one force sensor located near said at least one electrode, configured to measure the force applied by said electrode on said blood vessel tissue when said array body expands.

### Example 29

The electrode array of examples 1 or 2, further comprising at least one position sensor connected to said array body, configured to measure spatial location and/or orientation of said electrode array within said blood vessel.

### Example 30

The electrode array of example 6, wherein said contact point has an area of 1 mm²-3 mm².

### Example 31

The electrode array of examples 1 or 2, wherein expansion of said array body contacts less than 30% of said blood vessel inner tissue.

### Example 32

A method for EP measurements by electrodes placed within a blood vessel, comprising:
inserting an electrode array into a selected region within said blood vessel;
selecting at least one electrode to be used for EP measurements;
deploying at least one selected electrode out of at least two axially spaced-apart electrodes by moving a cylindrical sheath covering the said selected electrode in a deploying direction that will expose said selected electrode and allow said selected electrode to contact a tissue of said blood vessel; and
measuring EP parameters by said selected electrode.

### Example 33

The method according to example 32 comprising, determining if said selected electrode is in a desired location.

### Example 34

The method according to example 33 comprising deploying at least one additional electrode, by moving said cylindrical sheath further in said direction to allow said additional electrode to contact a different region of said tissue.

### Example 35

The method according to example 32 comprising:
collapsing said selected electrode by moving said cylindrical sheath in an opposite direction to said deploying direction, to cover said selected electrode;
re-positioning said electrode array within said blood vessel;
deploying at least one selected electrode by moving a cylindrical sheath covering the said selected electrode in a deploying direction that will expose said selected electrode and allow said selected electrode to contact a tissue of said blood vessel;
measuring EP parameters by said selected electrode.

### Example 36

A catheter device, comprising:
a steerable catheter body;
an electrode array of claim 2 positioned at a distal section of said catheter body;
at least one reference electrode positioned proximally to said electrode array;
wherein movement of at least one of said three spaced-apart flexible elements of said electrode array to a relaxed state, anchors said electrode array within a blood vessel.

### Example 37

The catheter device of example 36, wherein said electrode array is sized and shaped to be placed at least partially within the coronary sinus.

### Example 38

The catheter device of example 37 wherein said at least one reference electrode is positioned in a distance of at least 10 cm from said electrode array.

### Example 39

The catheter device of example 38, wherein said at least one reference electrode is shaped and sized to be positioned within the vena cava.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as measuring EP parameters might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a general flow chart describing the process of electrode array insertion, according to some embodiments of the invention;

FIG. 2 is a flow chart describing a method for electrodes deployment, according to some embodiments of the invention;

FIG. 3 is a block diagram depicting a system for EP measurements and electric field application, according to some embodiments of the invention;

FIG. 4 is a schematic view of a system for EP measurements and electric field application, according to some embodiments of the invention;

FIGS. 5A-5D are schematic views of an electrode array, according to some embodiments of the invention;

FIG. 5E is a schematic view of an electrode array within a blood vessel with a variable diameter, according to some embodiments of the invention;

FIG. 5F is a schematic view of an electrode array with flexible arms positioned along the electrode array main body, according to some embodiments of the invention;

FIG. 5G is an image of an electrode array with self-expandable elements, according to some embodiments of the invention;

FIG. 5H is an image of a catheter device, according to some embodiments of the invention;

FIGS. 6A-6B are schematic views of electrode array parts, according to some embodiments of the invention;

FIGS. 7A-7B are schematic views of a catheter handle, according to some embodiments of the invention;

FIGS. 8A-8B are schematic views of electrode arrays within the coronary sinus, according to some embodiments of the invention;

FIG. 8C is a schematic view of a catheter device with electrodes inside and outside the coronary sinus, according to some embodiments of the invention;

FIGS. 8D and 8E are schematic views of an electrode array with self-expandable elements positioned within a blood vessel with varying inner diameters, according to some embodiments of the invention;

FIG. 9 is a schematic view of signal conduction pathways in the heart, according to some embodiments of the invention;

FIGS. 10A-10B are schematic views of a helical electrode array when electrodes are exposed and when electrodes are covered, according to some embodiments of the invention;

FIG. 11 is a schematic view of a helical electrode array, according to some embodiments of the invention;

FIG. 12 is a schematic view of a stent-like electrode array, according to some embodiments of the invention;

FIG. 13A is a side perspective view of flexible arms, according to some embodiments of the invention;

FIG. 13B is an upper perspective view of contact points between electrodes and blood vessel inner tissue, according to some embodiments of the invention; and

FIGS. 14A and 14B are images depicting the results of a mapping procedure from within the coronary sinus, according to some embodiments of the invention.

### DESCRIPTION OF SPECIFIC EMBODIMENTS OF THE INVENTION

The present invention, in some embodiments thereof, relates to an electrode array and, more particularly, but not exclusively, to an electrode array configured to be inserted into the coronary sinus.

An aspect of some embodiments relates to a hollow electrode array configured to be inserted into blood vessels, comprising at least two spaced apart electrodes that can be optionally separately deployed to make contact with axially spaced-apart regions of the blood vessel inner surface. In some embodiments, the electrode array is configured to be inserted into conical blood vessels, for example the coronary sinus (CS). In some embodiments, when deployed, the electrodes apply a force against the blood vessel inner tissue by a flexible element that pushes the electrodes against the tissue. Optionally, the flexible element is a self-expandable element configured to radially expand, and to push the electrodes with a radial force against the blood vessel inner tissue. In some embodiments, the force applied by the electrodes on the blood vessel inner tissue allows contact with the tissue but does not cause an injury, for example tearing of the tissue. In some embodiments, expansion of the flexible element pushes at least one electrode against the blood vessel inner tissue with a force designed not to damage venous tissue. In some embodiments, the force that is applied by each electrode and/or each flexible element on the blood vessel inner surface is in the range of 2-30 gr. In some embodiments, a force sensor is positioned near at least one electrode and/or near at least one flexible element and is configured to measure applied by the electrode and/or flexible element on the tissue. Optionally, at least one force sensor is connected to a connecting shat between electrode segments, and is configured to measure the force applied by the connecting shaft on the blood vessel during the navigation process. In some embodiments, when deployed, each of the electrodes and/or each flexible element are electrically and/or mechanically isolated from at least some of the other electrodes. In some embodiments, when deployed, each of the electrodes and/or each flexible element is electrically/and or mechanically isolated from the rest of the electrodes. In some embodiments, each of the flexible elements is connected to the electrode array via a separate connector. In some embodiments, the separate connector allows expansion and collapse of each flexible element without any mechanical interaction with other flexible elements

In some embodiments, when the electrodes are deployed and make contact with the tissue, their contact point with the tissue is isolated from electrodes connected to other flexible elements. In some embodiments, when the electrodes are deployed they contact less than 40%, 30%, 20% or 10% of the blood vessel inner tissue. In some embodiments, when the flexible elements expand they contact less than 40%, 30%, 20% or 10% of the blood vessel inner tissue.

In some embodiments, the electrode array forms a distal part of a catheter. In some embodiments, the electrode array comprises, an elongated shaft for example a central channel for a guide wire or a stylet, and at least one electrode segment, which further comprises at least one flexible arm carrying at least one electrode. In some embodiments, each flexible arm is connected via a separate electrical and/or mechanical connection to a base ring of the electrode segment. In some embodiments, this separate connection allows each of the arms to move independently relative to the base ring and/or other electrode-carrying arms.

In some embodiments, each electrode-carrying arm can move between a closed conformation, where the arm is confined within a cylindrical sheath, to an open conformation, where the arm pushes at least one electrode against the blood vessel inner tissue. In some embodiments, each flexible arm is opened to a relaxed state with a maximal pre-determined acute angle. In some embodiments, each electrode segment along the electrode array is opened to a different radial distance for example segments that are located at a distal location of the electrode array are opened to a smaller radial distance compared to proximal segments that are opened to a wider radial distance. In some embodiments, the conical shape formed by the varying radial distances has a narrow distal end and a wider proximal end. In some embodiments, this conical shape allows to apply more uniform forces on the blood vessel inner surface when electrode array is positioned within conical shaped blood vessel sections, for example the CS that have a narrow distal end and a wider proximal end.

Optionally, flexible arms located at distal locations of the electrode array may have a less resilient spring behavior, which allows to apply a uniform force on the tissue as flexible arms located at a more proximal location. In some embodiments, each flexible arm is configured to move to the same or a smaller acute angle compared to a more proximal flexible arm. In some embodiments, the pre-determined acute angle allows collapsing of the flexible arms when they are pushed by the cylindrical sleeve. In some embodiments, the maximal acute angle of each arm is in the range of 0-90°, 0-40°, 30-90° for example 45°.

In some embodiments each electrode segment is designed to allow opening of the electrode carrying arms to a pre-determined maximal degree based on the diameter of the CS in adults in specific anatomical locations, for example near the CS ostium. In some embodiments, when the electrode carrying arms are opened to their relaxed state, and the electrode array is rotated, the arms form a circle with a diameter in the range of 6-12 mm, for example 6, 7, 8, 9, 10, 11 mm. In some embodiments, arms located at distal locations along the electrode array are opened at a relaxed state to an acute angle in the range of 2-30 degrees, and arms located at more proximal regions of the electrode array are opened to an acute angle in the range of 30-80 degrees. In some embodiments, the different acute angles formed by the flexible arms at their relaxed state, allows to anchor and/or deploy selected electrodes within conical sections of the CS, for example the section of the CS which is closer to the CS ostium. In some embodiments, deploying selected electrodes in different regions of the CS allows directional EP measurements from different radial and/or axial locations in the blood vessel.

In some embodiments, an electrode segment contains at least one connecting shaft for connecting the base of the electrode segment with the base of the following segment and/or the previous segment. In some embodiments, the connecting shaft is an elongated shaft connecting the electrode segments. In some embodiments, the connecting shaft proximal end is positioned proximal to the most proximal electrode segment, and the connecting shaft distal end is connected to a cap at the distal tip of the electrode array. In some embodiments, the connecting shaft is configured to be bent by a user of the catheter to allow the insertion of the electrode array into a blood vessel. In some embodiments, the connecting shaft is at least 2 times more resilience than the flexible arms when a force is applied from a distance of at least 2 mm in an angle of at least 80 degrees. Alternatively, the flexible arms are at least 2 times more resilience than the connecting shaft when a force is applied from a distance of at least 2 mm in an angle of at least 80 degrees. In some embodiments, the connecting shaft is electrically isolated from any electrode connected to the flexible arm. In some embodiments, each electrode array comprises 1-10 electrode segments, for example 2, 3, 4, 5 or 6 electrode segments. In some embodiments, each electrode segment comprises 1-7 flexible arms, for example 2, 3, 4 or 5 flexible arms. In some embodiments, each flexible arm carries 1-4 electrodes, for example 2, 3 or 4 electrodes. In some embodiments, each electrode array comprises 2-50 electrodes, for example 2-15 or 20-30 electrodes.

In some embodiments, the electrode array comprises a hollow shaft connecting the electrode segments. In some embodiments, a PCB component for each segment, carrying wiring and electrodes is positioned within the hollow shaft.

Alternatively, a single PCB component carrying wiring and electrodes to at least two electrode segments is positioned within the hollow shaft. In some embodiments the distal end of each PCB segment carries the electrodes and is attached to the flexible elements of the electrode segment.

In some embodiments, the electrode array comprises at least two electrodes connected to a hollow helical body, and a cylindrical sheath configured to confine the electrodes in a closed conformation when the electrodes are collapsed. In some embodiments, when the cylindrical sheath is retracted, the helical body self-expands to an open conformation. In some embodiments, in an open conformation the helical body has a fixed diameter. Alternatively, in an open conformation the helical body has a varying diameter. Optionally, in an open conformation the helical body distal section has a smaller diameter compared to the proximal section of the helical body.

In some embodiments, when the helical body radially expands, it pushes at least one electrode connected to its outer surface against the blood vessel wall. In some embodiments, when the helical body radially expands it acquires a conical open conformation. Alternatively, when the helical body radially expands, it acquires a tubular open conformation with a ratio of at least 1:3 between the diameter and the length of the tubular conformation. In some embodiments, the electrode array, for example when the electrodes or the self-expandable elements are collapsed, is in the size of at least 5 French, for example 6, 7, 9 French. In some embodiments, the catheter device is in the size of at least 5 French, for example 6, 7, 9 French.

An aspect of some embodiments relates to an electrode array comprising at least two electrodes arranged along the array body at pre-determined locations to allow contact with at least two axially and/or radially spaced apart tissue regions of the blood vessel inner tissue. In some embodiments, the electrode array forms a distal end of a catheter comprising at least two electrodes arranged along the electrode array body at pre-determined locations to allow contact with at least two axially and/or radially spaced apart tissue regions of the blood vessel, for example with the proximal section of the CS, near the CS ostium and with a more distal section of the CS. In some embodiments, the electrodes are localized on the electrode array based on different anatomical properties of the blood vessel tissue regions, for example the distance between the two regions, their distance from the CS ostium, their relative angle etc. In some embodiments, the electrodes are localized to allow EP measurements and/or to apply an electric field to signal conduction pathways of the heart. Optionally, the electrodes are localized to allow EP measurements and/or to apply an electric field to at least two different signal conduction pathways of the heart.

In some embodiments, when at least one flexible arm is opened it pushes at least one electrode with a force anchors the electrode and/or the electrode array in a specific contact point with the tissue. In some embodiments, when more flexible arms are opened the electrode remains at its original contact point.

In some embodiments, each flexible arm contains at least 2 electrodes at the distal end of the flexible arm. In some embodiments, each of the two electrodes can measure EP values compared to the other electrode of the same flexible arm and compared to electrodes of other flexible arms of the electrode array.

An aspect of some embodiments relates to controlling which electrodes of an electrode array, will be in contact with a blood vessel tissue, for example CS tissue. In some embodiments, the electrodes are placed within a cylindrical sheath configured to cover the electrodes as the electrode array is introduced into the CS lumen.

Optionally, the cylindrical sheath covers the electrodes when the electrode array is retracted from the CS lumen. In some embodiments, when the electrode array is placed in a desired location, the cylindrical sheath is partially retracted to deploy at least some of the distally located electrodes. In some embodiments, the cylindrical sheath is further retracted to deploy additional, more proximal electrodes.

Alternatively, the cylindrical sheath is pushed forward to deploy the most proximal electrode or electrode set. Optionally, the cylindrical sheath is rotated to deploy a selected electrode or electrode set through a window in its outer surface.

An aspect of some embodiments relates to protecting vein walls, when moving an electrode array, for example the distal end of a catheter, inside the vein lumen. In some embodiments, an electrophysiological (EP) catheter comprises an electrodes cover configured to protect the CS inner wall by separating the electrodes from the CS wall. In some embodiments the electrode cover separates between the electrodes and the CS wall during electrode array insertion and retraction. In some embodiments when the electrode array is in a desired place, the electrodes cover is removed to allow the electrodes to contact the CS tissue.

An aspect of some embodiments relates to EP mapping of signal conduction in the by allowing at least two selected electrodes out of a plurality of electrodes, to make contact with at least two different radial and/or axial locations of a blood vessel inner wall, for example the CS inner wall. In some embodiments, the electrodes sense signals propagating from the atria to the ventricle, for example through the atrioventricular node (AV node) or AV node extensions. In some embodiments, at least one electrode is deployed to make contact with the CS inner surface near the CS ostium. Optionally, at least one electrode is a reference electrode, and is deployed for example outside of the CS. In some embodiments, the reference electrode is positioned within the vena cava. In some embodiments, a user selects which electrode or electrodes will be used as a reference. In some embodiments EP mapping comprises application of an electric field through at least one selected electrode out of the deployed electrodes.

In some embodiments, EP measurements of at least one selected electrode will be used to generate high-dense mapping and high resolution representation of electro-anatomical information in the CS and its surroundings. In some embodiments, at least one selected electrode will be used to generate a stable and/or a reproducible signal that will be used for example for construction of electrical activation maps.

Optionally, multiple electrodes will be deployed and used to allow multiple points and angles for reference for better synchronization of local activation time in the heart relative to a constant, stable, CS signal. Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Method for Insertion of a Catheter

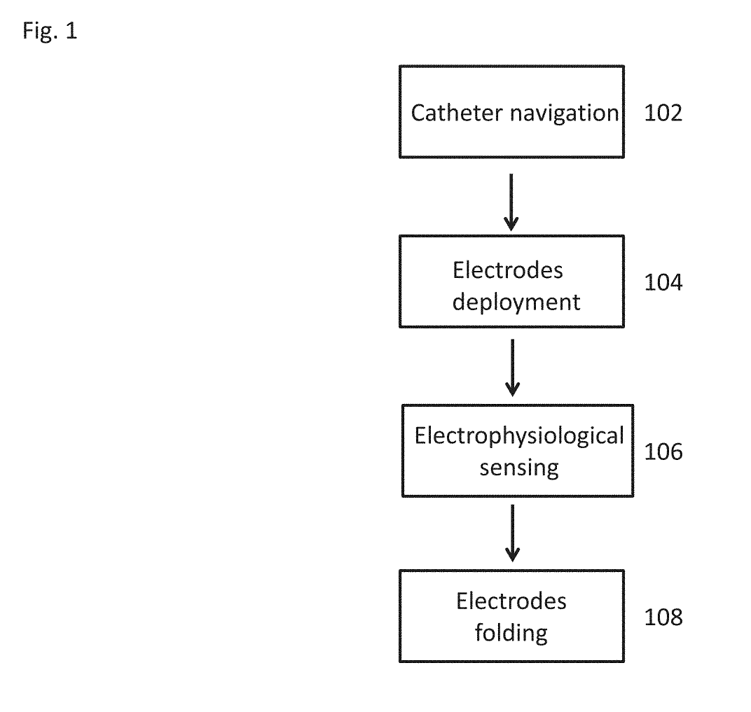

According to some embodiments, an electrode array, for example provided at the distal end of a catheter is sometimes used to measure and record the electrical activity of the heart. Alternatively, an electrode array is used to deliver an electric field to a tissue. In some embodiments, insertion of an EP catheter is used to diagnose arrhythmia. Optionally, an EP catheter is inserted prior to an electrophysiological intervention, for example an ablation procedure. Reference is now made to FIG. 1 depicting a process for insertion of an EP catheter, according to some embodiments.

According to some exemplary embodiments, an electrode array, for example provided at the distal end of a catheter is inserted through blood vessels travelling to the heart. In some embodiments, the distal end of the catheter is navigated to a desired location in the patient's heart, for example the CS blood vessel at 102. In some embodiments, the electrode array of the catheter is inserted to the right atrium and through the CS ostium into the CS lumen. In some embodiments, the catheter is at least partly made from a flexible material which allows it to bend in several points along the catheter body. In some embodiments, bending the catheter body allows its navigation from the right atria and into the CS ostium.

Optionally, a guide wire, for example a J-shaped guide wire is introduced to the blood vessel prior to insertion of the catheter. In some embodiments, the catheter is pushed and guided by the guide wire to its desired location

According to some exemplary embodiments, once the catheter is inside the blood vessel, it is navigated to a desired location within the blood vessel and deploys at least one electrode at 104. In some embodiments, determining which electrode to deploy is based on its distance from signal conduction pathways and/or its ability to make contact with the blood vessel tissue, for example the CS inner wall. In some embodiments, in order to deploy a selected electrode or a selected electrode set, a cylindrical sheath covering the selected electrode/s is removed. In some embodiments, when the sheath is removed, the electrode/s can make contact with the blood vessel wall. In some embodiments, when the sheath is removed a flexible arm is allowed to expand and to push at least one electrode against the blood vessel inner wall. Alternatively, when the sheath is removed, the electrode array main body which carries the electrodes expands into an open helical conformation which pushes at least some of the electrodes against the blood vessel wall. In some embodiments, in order to deploy a selected electrode or a selected electrode set, the cylindrical sheath is rotated until the selected electrode or electrode set is exposed through a window in the sheath's outer surface.

Optionally, electrodes are deployed by retracting a stylet which travels inside the electrode array main tube. In some embodiments, removing the stylet from regions of the electrode array main tube allows these regions to self-expand, for example to a helical formation, and to push at least one electrode against the blood vessel wall. In some embodiments, to collapse the at least one electrode, the stylet is pushed back to its place and forces the electrode array main tube to acquire a closed conformation.

According to some exemplary embodiments, when the selected electrode is deployed and is in contact with the blood vessel tissue, the electrode starts to sense EP properties of the cardiac tissue at 106. In some embodiments, the EP properties are related to signals conducted between the atria and ventricles of the heart. Optionally, measuring EP properties further includes delivering of an electric field to the blood vessel tissue.

According to some exemplary embodiments, when sensing the EP properties is completed, the selected electrodes are collapsed back to a closed conformation at 108. In some embodiments, the electrodes are collapsed by moving the cylindrical sheath to cover the electrodes. In some embodiments, when the electrodes are covered, the catheter can be retracted from the blood vessel.

### Exemplary Electrodes Deployment Process

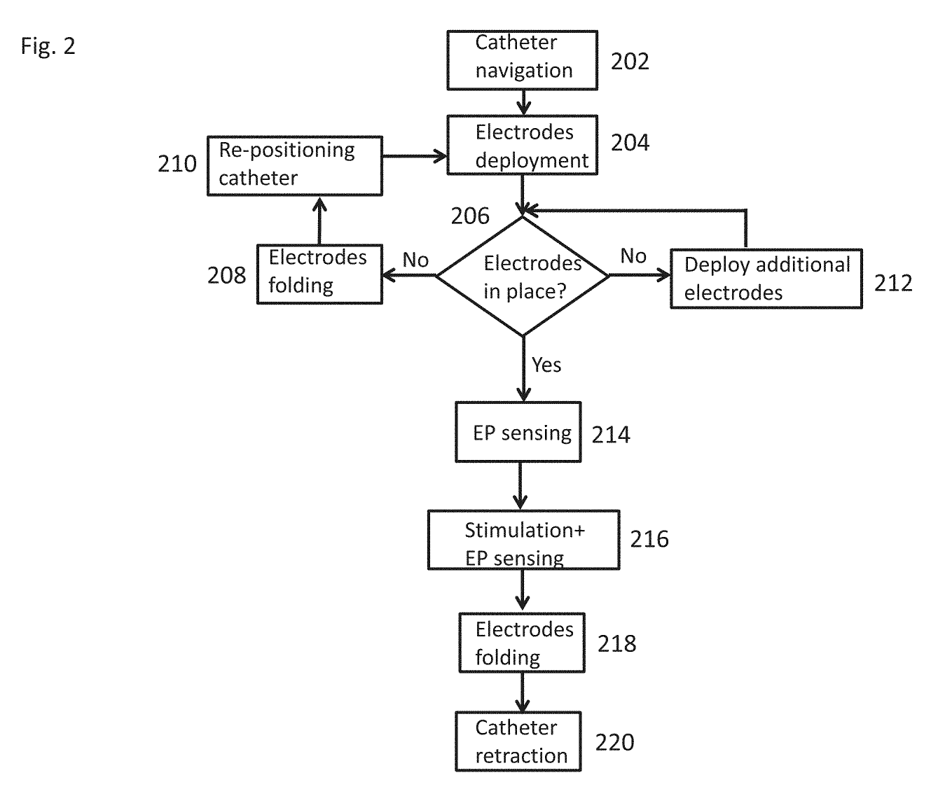

According to some embodiments, when an electrode array, for example provided at the distal end of a catheter is navigated to a desired location within a blood vessel, at least one electrode is deployed. In some embodiments, a user controlling the catheter decides which electrode or set of electrodes to deploy Reference is now made to FIG. 2 depicting an electrodes deployment process, according to some embodiments of the invention.

According to some exemplary embodiments, a catheter is navigated through blood vessels leading to the heart, until a desired location is reached at 202. In some embodiments, the desired location is a blood vessel, for example the CS.

According to some exemplary embodiments, once the electrode array of the catheter is inserted to the blood vessel, it is further navigated to a desired location where at least one electrode is deployed at 204. In some embodiments, when a selected electrode is deployed, it makes contact with a selected tissue or region of the blood vessel. In some embodiments, the selected tissue and/or region are pre-determined. In some embodiments, a selected electrode is deployed based on the distance of its contact region with the blood vessel tissue and a target region of the heart. Alternatively, a selected electrode is deployed based on the distance of its contact region with the blood vessel tissue and signal conducting pathways of the heart.

According to some exemplary embodiments, after an electrode is deployed, a catheter user determines whether the electrode makes contact with the desired tissue and/or region of the blood vessel at 206. In some embodiments, if the electrode is not in contact with the correct tissue and/or region, at least one additional electrode or electrode set is deployed at 212. In some embodiments, to deploy at least one additional electrode, the cylindrical sheath covering the additional electrode is removed, to allow the additional electrode to make contact with the blood vessel tissue. In some embodiments, when the cylindrical sheath is removed, an additional section of the catheter tube shifts to an open conformation, where the catheter pushes the deployed electrodes against the blood vessel inner wall.

In some embodiments, if the electrode is not in the desired place at 206, then the cylindrical sheath is moved to separate between the electrode and the tissue, which causes the electrode to collapse at 208. In some embodiments, when an electrode is collapsed, it shifts from an open conformation to a closed conformation where it is confined within the cylindrical sheath. In some embodiments, after the electrode is collapsed at 208, the catheter can be either retracted or pushed to reach a new position within the blood vessel lumen at 210. In some embodiments, to reach a new position within the blood vessel lumen, the catheter is configured to bend in at least one point along its structure. In some embodiments, after re-positioning the catheter at 210, a selected electrode is deployed at the new position, as previously explained at 204.

According to some exemplary embodiments, if the deployed electrode or electrodes set is in contact with the desired tissue of the blood vessel, they start to sense EP parameters of the cardiac tissue, and perform EP measurements at 214. In some embodiments, the signals acquired from the electrodes are used to construct an EP map representing local activation times, local voltage map or any other physical parameter which is found discriminative between adjacent points on the catheter's electrode array. In some embodiments, this map is used to determine the approximate location of conduction or other EP abnormality requiring treatment by means such as radio frequency (RF) ablation. In some embodiments, each electrode is separately connected to an electrically conductive wire travelling from the electrode contact through the catheter handle until it reaches a connector of an EP measuring unit located outside of the patient's body. In some embodiments, although several electrodes are exposed and in contact with the blood vessel tissue, only selected electrodes of the exposed electrodes are used for EP sensing. In some embodiments the electrodes are in contact with blood vessel tissue regions that are spaced apart and/or have a different radial position.

According to some exemplary embodiments, the deployed electrode or electrodes set is used to apply an electric field to the blood vessel tissue at 216. In some embodiments, the electric field is applied through at least one of the deployed electrodes. In some embodiments, the deployed electrode or a set of deployed electrodes is used to deliver RF energy to the blood vessel tissue, for example during RF ablation procedures. In some embodiments, at least one selected electrode of the deployed electrodes is used for EP sensing, and at least one additional electrode out of the deployed electrodes is used for electric field application. In some embodiments, the deployed electrode used for electric field application is selected based on its location within the blood vessel lumen and/or based on the location of its contact point with the blood vessel tissue. In some embodiments, in order to analyze the effect of the applied electric field, at least one of the deployed electrodes is used for EP sensing post electric field application. In some embodiments, in response to the EP sensing post electric field application, an additional electric field is applied using the same deployed electrode or using a different deployed electrode or electrodes set.

According to some exemplary embodiments after the EP sensing and/or the electric field application is finished, the deployed electrodes are collapsed at 218. In some embodiments the at least one deployed electrode is collapsed by changing its conformation from an open conformation to a closed conformation. In some embodiments, to change the conformation of the at least one deployed electrode, a cylindrical sheath is pushed to separate between the at least one deployed electrode and the blood vessel tissue. Alternatively, to separate the at least one electrode from the blood vessel tissue, the cylindrical sheath is rotated and is pushed between the electrode and the tissue.

According to some exemplary embodiments, after the electrodes are collapsed, the catheter is retracted from the blood vessel lumen at 220. In some embodiments, the catheter is retracted from the lumen of a first blood vessel and is navigated to enter the lumen of a second blood vessel. Alternatively, the catheter is retracted from the first blood vessel lumen and from the patient's body.

According to some exemplary embodiments, electrodes deployment, for example as described at 204 at selected locations in the CS will allow high-density EP measurements and/or high resolution representation of electro-anatomical information in the CS, and its surroundings. In some embodiments, deployment of the electrode array electrodes at different locations within the CS, and optionally, at least one electrode outside the CS, will allow multiple points and angles for reference, allowing to measure more accurately synchronization of local activation time in the heart relative to a constant, stable, CS signal. In some embodiments, the reference electrode is positioned within the vena cava, for example within the superior vena cava or within the inferior vena cava. Optionally, the reference electrode contacts the inner wall of the vena cava.

Exemplary System for EP Sensing and/or Electric Field Application

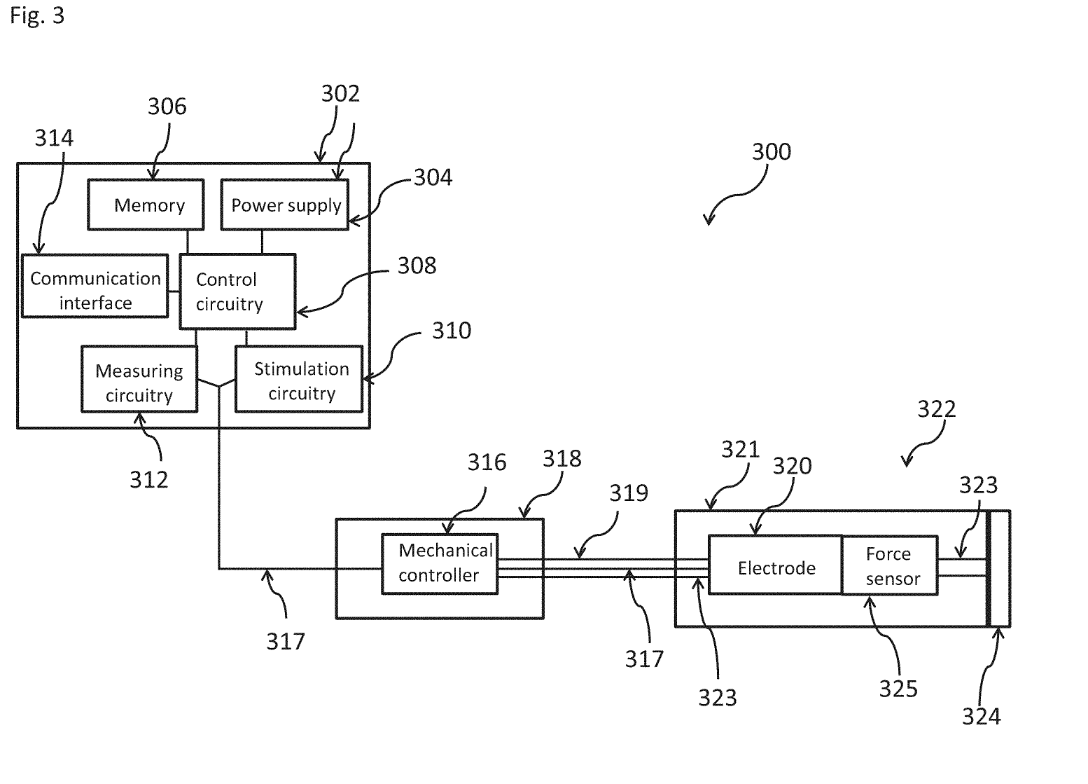

According to some exemplary embodiments, a system for EP sensing and/or electric field application comprises a catheter with at least one electrode configured to be placed within a blood vessel, for example the CS, a catheter handle for navigating the catheter and an EP measuring and electric field application device. In some embodiments, the catheter handle and the EP measuring and electric field application device are located outside the patient's body. Reference is now made to FIG. 3 depicting a system for EP sensing and/or electric field application, according to some embodiments of the invention.

According to some exemplary embodiments, a system for EP sensing and/or electric field application 300 comprises an electrode array 322, connected to a catheter handle 318 via a flexible tube 319 and an EP measuring and stimulating device 302.

In some embodiments, electrode array 322 comprises a central body 323 which has at least one electrode 320 connected to it, and a cylindrical sheath 321 covering the at least one electrode 320 when the electrode is in a closed conformation.

In some embodiments, central body 323 comprises a helical elongated tube which has at least one electrode connected to its outer surface. Alternatively, the helical elongated tube has a plurality of electrodes positioned at desired locations along the tube.

In some embodiments, electrode 320 is connected to central body 323 by a flexible arm, configured to move electrode 320 from a closed conformation to an open conformation. In some embodiments, when cylindrical sheath 321 covers electrode 320, it prevents it from contacting the blood vessel tissue. In some embodiments, when cylindrical sheath 321 covers electrode 320, it pushes the flexible arm to a closed conformation, where both the flexible arm and the electrode are confined within the cylindrical sheath. In some embodiments, when electrode array 322 is navigated into a blood vessel lumen, for example the CS, cylindrical sheath 321 covers the at least one electrode 320, and forces it to be in a closed conformation.

In some embodiments, when electrode array 322 is positioned within a desired location within the blood vessel lumen, cylindrical sheath 321 is moved or rotated to expose the at least one electrode 320 to the blood vessel tissue. Optionally, when cylindrical sheath 321 is moved or rotated, the flexible arm of a selected electrode or electrodes set expands into an open conformation and pushes the at least one electrode 320 against the blood vessel tissue.

According to some exemplary embodiments, at least some of electrode array 322 electrodes are configured to deliver radio frequency energy to the blood vessel tissue, for example during RF ablation procedures.

According to some exemplary embodiments, electrode array 322 comprises at least one force sensor, for example force sensor 325, to sense the force applied by the at least one electrode on the blood vessel tissue. In some embodiments, the force sensor is located at the contact point of at least one electrode with the blood vessel tissue. Optionally, the force sensor is located on a flexible element carrying the electrode and/or the electrode array central body, for example central body 323.

In some embodiments, the distal end of a catheter, for example electrode array 322 comprises a strain sensor for sensing the strain of the electrode array, and or the strain of electrode array central body 323.

According to some exemplary embodiments, each electrode 320 is connected via an electrical conductive wire 317 to an EP measuring and stimulating device 302.

In some embodiments, electrode 320 is a unipolar electrode and senses EP parameters of the tissue. In some embodiments, a catheter cap 324 and/or central body 321 comprises at least one electrode, configured to sense EP parameters of the tissue when. In some embodiments, this electrode is used as a reference electrode to the at least one electrode 320.

According to some exemplary embodiments, electrode array 322 is made from a flexible material which allows it to bend. In some embodiments, electrode array central body 323 and/or the flexible arm coupled between the at least one electrode 320 and central body 323 are made from elastic or super-elastic materials, for example Nitinol.

According to some exemplary embodiments, catheter handle 318 comprises a mechanical controller 316, configured to control the navigation of electrode array 322 into a desired blood vessel by bending electrode array 322 in at least one point along its structure. In some embodiments, mechanical controller 316 is configured to expose at least one selected electrode, for example electrode 320 to the blood vessel tissue by moving cylindrical sheath 321 either backward or forward. In some embodiments, mechanical controller 316 rotates cylindrical sheath 321 to expose at least one selected electrode. In some embodiments, to collapse at least one selected electrode, mechanical controller 316 moves cylindrical sheath 321 to cover the at least one selected electrode. In some embodiments, mechanical controller 316 controls the deployment and/or collapsing of a selected electrode or electrodes set and/or at least one flexible element by moving and/or rotating cylindrical sheath 321.

According to some exemplary embodiments, EP measuring and stimulating device 302 comprises a measuring circuitry 312 and/or a stimulation circuitry 310 which are connected to electrode 320 via wire 317. In some embodiments, each electrode of electrode array 322, which comprises a plurality of electrodes, is separately connected to measuring circuitry 312 and/or to stimulating circuitry 310.

In some exemplary embodiments of the invention, the electrodes, for example electrode 320 are spaced apart, for example, 1 mm, 2 mm, 3 mm or smaller or intermediate distances. Optionally, the spacing is uniform. In some embodiments, the spacing is non-uniform, for example, a smaller spacing provided in areas closer to signal conduction pathways of the heart. Optionally or alternatively, to axial spacing, circumferential spacing may be provided, for example, 20 degrees, 40 degrees, 90 degrees, 120 degrees, 180 degrees and smaller, intermediate and/or larger spacing. In some cases (e.g., helical designs) spacing may be simultaneously axial (and/or along the surface of the catheter) and angular.

In some exemplary embodiments of the invention, even at a same axial location, multiple angular locations may be provided, for example, 2, 3, 4, 5 or larger numbers of electrodes, each aimed in a different direction.

In some exemplary embodiments of the invention, an electrode, for example electrode 320, has an area of between 0.1 mm square and 5 mm square, for example, between 1 and 4 mm square. Optionally, an electrode is generally rectangular or circular with a maximal extent of between 0.1 and 4 mm, for example, between 0.5 and 2 mm. In some exemplary embodiments of the invention, an electrode is ring shaped with a width of, for example, between 0.1 and 5 mm.

In some exemplary embodiments of the invention, an axial extent of electrodes (along which electrodes are located) is between 0.1 and 70 mm, for example, between 2 and 10 mm or between 4 and 30 mm. In some embodiments, electrodes for example electrode 320 is a unipolar electrode. In some embodiments, each of the electrodes is separately connected to device 302. Alternatively, some of the electrodes are electrically inter-connected to device 302.

In some embodiments, measuring circuitry 312 is configured to measure EP parameters delivered from at least one deployed electrode. In some embodiments the deployed electrode selected to measure EP parameters is in contact with the blood vessel tissue. In some embodiments, stimulation circuitry 310 is configured to deliver an electric field to the blood vessel tissue through at least one exposed electrode. In some embodiments, the exposed electrode used to deliver an electric field is in contact with the blood vessel tissue. In some embodiments, stimulation circuitry generates RF energy, to be delivered to the blood vessel tissue through at least one deployed electrode of electrode array 322.

According to some exemplary embodiments, EP measuring and stimulating device 302 comprises a control circuitry 308 connected to stimulation circuitry 310 and/or measuring circuitry 312. In some embodiments, control circuitry 308 determines whether to measure EP parameters using measuring circuitry 312 or to apply an electric field using stimulating circuitry 310. Alternatively, control circuitry 308 determines whether to measure EP parameters using a selected electrode or set of electrodes, while applying an electric field to the blood vessel tissue using a different electrode or set of electrodes. In some embodiments, control circuitry 308 is configured to determine which electrode or electrode set to use out of the deployed electrodes. In some embodiments, control circuitry 308 determines which electrode to use based on its contact with the blood vessel tissue, the type of the blood vessel tissue which is in contact with the electrode, the distance between the electrode and signal conduction pathways of the heart and/or the distance of the electrode from anatomical features of the blood vessel, for example the CS ostium.

In some embodiments, control circuitry 308 determines which electrode or electrode set to use for application of an electric field based on EP measurements.

According to some exemplary embodiments, control circuitry 308 is connected to a communication interface 314 configured to transmit and/or receive log files, and/or operation protocols and/or EP measurements to a computer and/or a handheld device. In some embodiments, communication interface 314 communicates with the computer and/or the handheld device via wired and/or wireless means.

According to some exemplary embodiments, control circuitry 308 is connected to a memory component 306, configured to store EP parameters and/or EP measurements. In some embodiments, memory component 306 is configured to store operation protocols of EP measuring and stimulating device 302. In some embodiments, memory component 306 is configured to store electric field application parameters.

According to some exemplary embodiments, EP measuring and stimulating device 302 comprises a power supply unit, connected to control circuitry 308 configured to supply electric power to device 302 and/or catheter handle 318 and/or catheter 321.

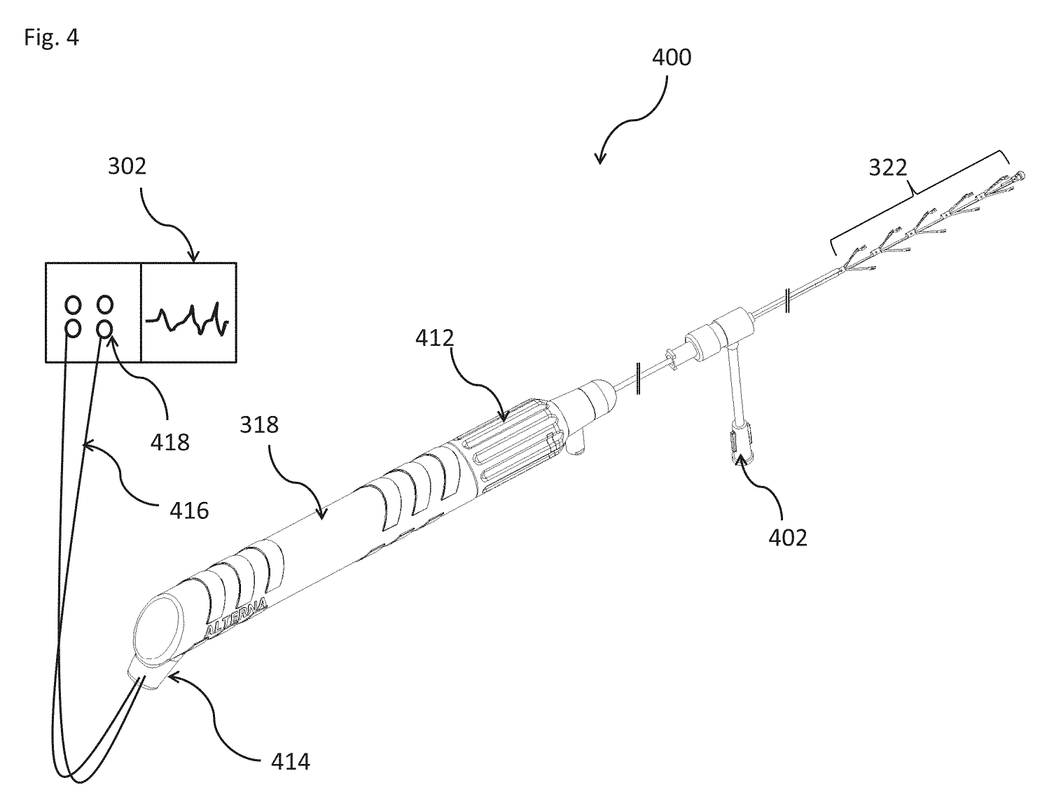

Reference is now made to FIG. 4 depicting a system for EP sensing and/or electric field application, according to some embodiments of the invention.

According to some exemplary embodiments, system for EP sensing and/or electric field application 400 comprises an electrode array 322, a catheter handle 318, and an EP measuring and stimulating device 302. In some embodiments, an input member 402 is coupled between electrode array 322 and catheter handle 318 and is configured to allow the delivery of liquids through electrode array 322 to the heart tissue.

In some embodiments, catheter handle 318 comprises a control component 412 configured to control catheter bending to allow its navigation into selected blood vessels. Optionally, control component 412 is configured to control cylindrical sheath 321 movements, in order to deploy or collapse at least one selected electrode or electrodes set. In some embodiments, catheter handle 318 comprises a wiring output 414 configured to allow at least one wire 416 connected to at least one electrode of electrode array 322 to pass through catheter handle 318 and to connect the electrode with EP measuring and stimulating device, for example device 302. In some embodiments, each electrode of electrode array 322 is connected via a different wire 416 to device 302, through connector 418. Alternatively, wire 416 is connected to at least two electrodes of electrode array 322.

According to some exemplary embodiments, electrode array 322 is inserted into the CS and sense electrical activity at specific locations along the inner tissue of the CS. In some embodiments, the sensed electrical activity is delivered to an electro-anatomical mapping device, which generates high-dense mapping and/or high-resolution representation of electro-anatomical information in the CS and its surroundings, based on the sensed electrical activity. In some embodiments, positioning of electrode array 322 at selected locations within the CS allows stable and reproducible measurements of the electric signal, which is then used as a reference signal to construct electrical activation maps.

### Exemplary Electrode Arrays

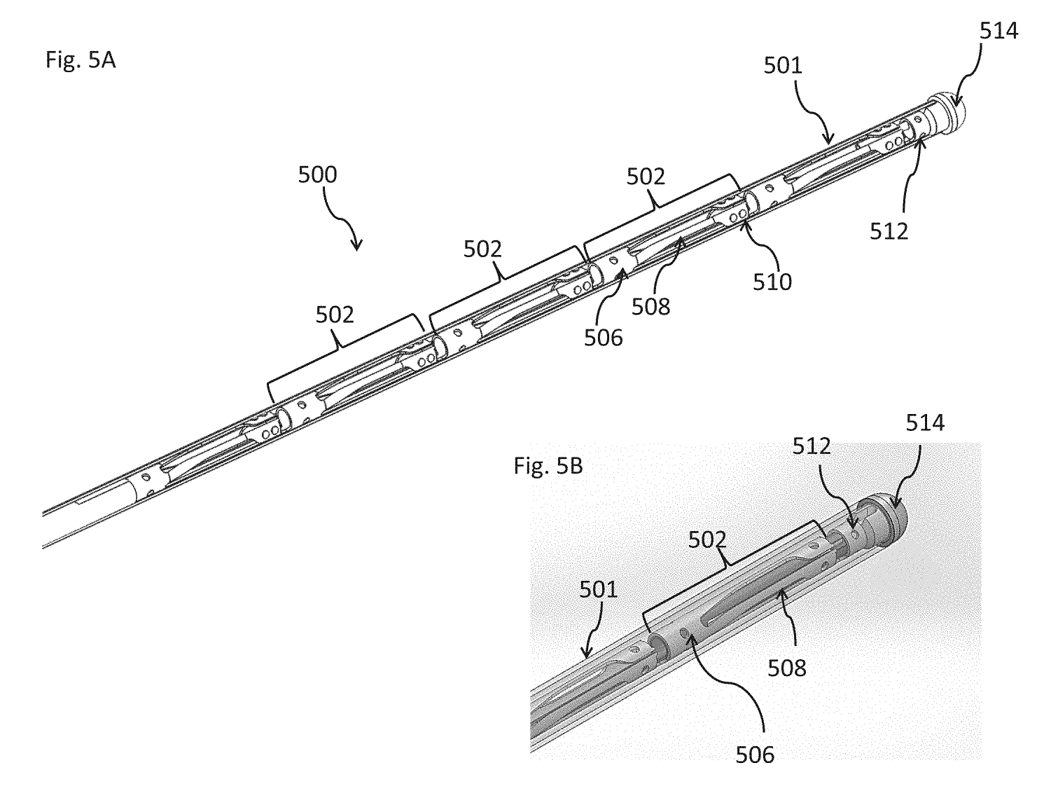

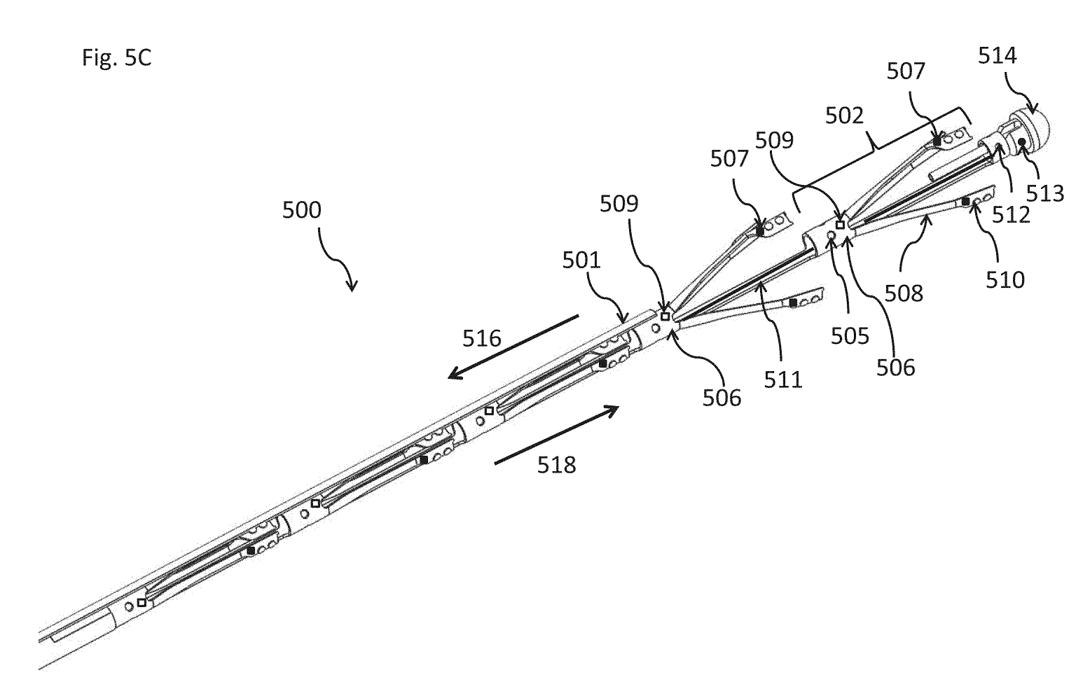

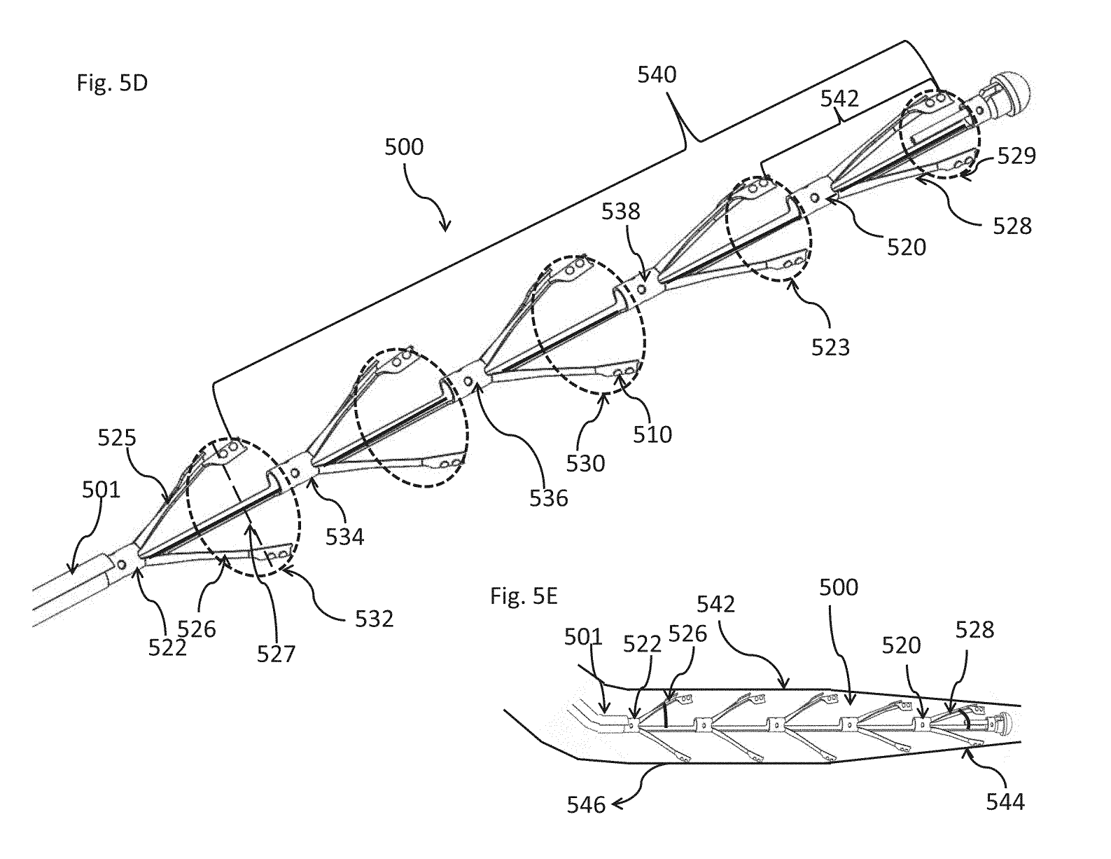

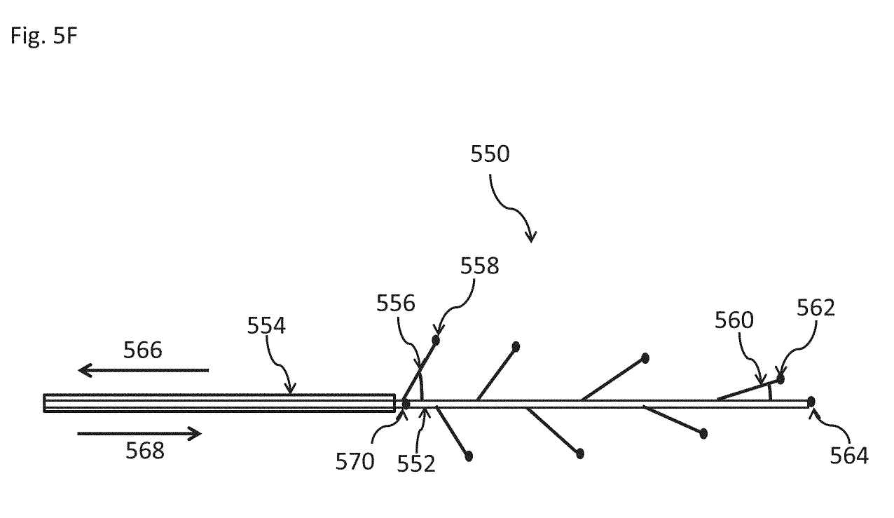

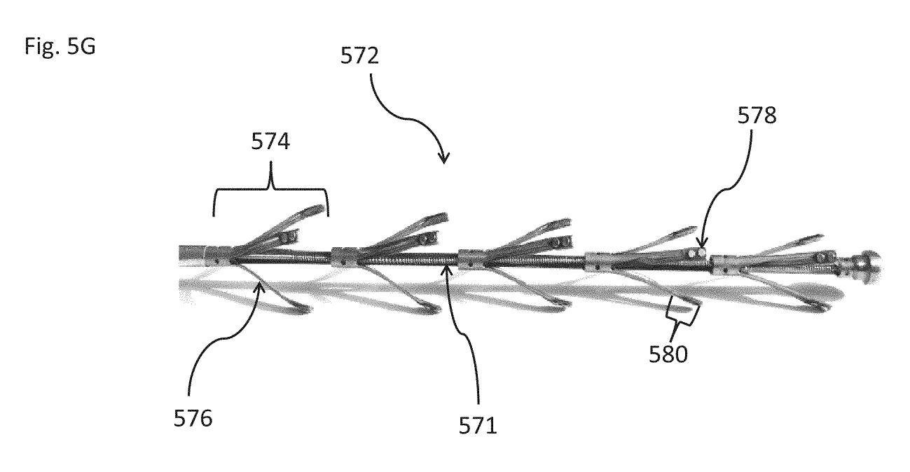

According to some exemplary embodiments, when an electrode array is navigated to a desired location through different blood vessels, its electrodes are in a closed conformation, and are enclosed within a cylindrical sheath. Reference is now made to FIGS. 5A and 5B depicting an electrode array with electrodes in a closed conformation, according to some embodiments of the invention.

According to some exemplary embodiments, electrode array 500 comprises at least one electrode segment 502 enclosed within cylindrical sheath 501, when electrode array 500 is navigated. In some embodiments, electrode array 500 comprises a plurality of axially spaced-apart electrode segments, for example 3-10 or 3-6 electrode segments. In some embodiments, electrode array 500 further comprises a cap 514 at the most distal end of the electrode array, configured to protect blood vessel tissue during electrode array navigation process.

According to some exemplary embodiments, each electrode segment 502 further comprises an electrode segment base ring 506 which has at least one flexible arm 508 connected to it. In some embodiments, each arm 508 comprises at least one electrode 510 near the distal end of the arm. In some embodiments, a plurality of flexible arms, for example 2-10 or 2-5 flexible arms are connected to each ring, for example ring 506 in a separate electrical and/or mechanical connection. In some embodiments, cap 514 comprises at least one cap electrode 512 near the distal end of electrode array 500. In some embodiments at least one cap electrode 512 and/or at least one electrode connected to at least one ring, for example ring 506 serve as a reference electrode.

Reference is now made to FIG. 5C depicting an electrode array, for example an electrode array 500 during an electrodes deployment process, according to some embodiments of the invention.

According to some exemplary embodiments, when electrode array 500 arrives to a desired location within a blood vessel, for example the CS, cylindrical sheath is moved in direction 516 to expose at least one selected electrode or a selected arm, for example arm 508. Alternatively, cylindrical sheath 501 is turned to expose at least one selected electrode or a selected arm 508 through a window in sheath 501 surface.

Optionally, cylindrical sheath 501 is moved and turned to allow exposure of at least one selected electrode or at least one selected arm 508. In some embodiments when cylindrical sheath 501 is moved in direction 516, electrode segments located near the distal end of electrode array 500 are exposed first. In some embodiments, if exposure of additional electrodes is desired then cylindrical sheath can be further moved in direction 516 to expose more electrode segments and/or at least one additional electrode.

According to some exemplary embodiments, the deployed electrode, for example electrode 510 is configured to deliver RF energy to the blood vessel tissue, for example during RF ablation procedures.

According to some exemplary embodiments, if electrode array 500 needs to be re-positioned, then cylindrical sleeve 501 is moved in direction 518 to allow collapsing of at least one flexible arm, for example arm 508, to a closed conformation.

Alternatively, moving cylindrical sleeve 501 in direction 518 allows collapsing of at least one electrode, for example electrode 510 to a closed conformation.

According to some exemplary embodiments, electrode segment 506 comprises at least one flexible arm 508 carrying at least one electrode 510, and a connecting shaft 511 to connect each electrode segment to the following electrode segment. In some embodiments, connecting shaft 511 connects the base rings of adjacent electrode segments. In some embodiments, when the flexible arms are in a closed conformation they form together with the connecting shaft 511 and inner tube that has a smaller diameter compared to the diameter of cylindrical sleeve 501.

According to some exemplary embodiments, some electrodes segments, for example electrode segment 502, comprise at least one electrode 505 at their base ring, for example base ring 506. Optionally, the at least one base ring electrode serves as a reference electrode.

According to some exemplary embodiments, electrode array 500 comprises at least one position sensor, for example position sensor 513 at its distal tip. Optionally, a position sensor is located on at least one base ring 506, and/or at least one flexible element, for example flexible arm 508. Alternatively, the position sensor is located at the distal end of flexible arm 508, adjacent to at least one electrode. In some embodiments, the at least one position sensor is configured to sense and transmit the position of the electrode array and/or at least one electrode to an EP device that is used to track the position of the electrode array and/or at least one electrode. In some embodiments, the EP device uses electromagnetic fields to determine the spatial location and/or rotation of the electrode array and/or the at least one electrode.

According to some exemplary embodiments, electrode array 500 comprises at least one force sensor, for example force sensor 507 connected to at least one flexible element of the catheter, configured to measure the force applied by the at least one flexible element on the blood vessel tissue. In some embodiments, the force sensor is connected to at least one flexible arm, for example flexible arm 508. In some embodiments, electrode array 500 comprises at least one strain sensor, configured to measure the strain of the catheter. In some embodiments the strain sensor, for example strain sensor 509 is connected to base ring 506. Alternatively, the strain sensor is connected to the electrode array central body.

Reference is now made to FIG. 5D depicting an electrode array, for example the distal end of a catheter with electrodes in an open orientation, according to some embodiments of the invention.

According to some exemplary embodiments, an electrode array, for example electrode array 500 comprises a plurality of axially spaced apart segments, between 1-10 segments, for example 3, 4, 5 segments, connected to each other by a central shaft.

In some embodiments, each of the segments comprises at least one flexible element, for example, flexible arm 526 configured to move between a collapsed state to a fully expanded and relaxed open state. In some embodiments, each of the five segments of electrode array 500 comprises at least 3 flexible elements, for example flexible arms 526 and 525. In some embodiments, each flexible element of the same segment is located 20-120 radial degrees, for example 25-55 or 80-99 radial degrees from the other flexible elements of the segment. Alternatively, the radial distance between each flexible element of the same segment is between 15-120 degrees. In some embodiments, the segments, the shaft and the flexible elements are made from elastic or super-elastic materials, for example Nitinol. In some embodiments, at least one of the flexible elements has a window at its distal ending to allow positioning of at least one electrode. In some embodiments, the electrode array comprises a flexible PCB carrying at least one electrode is positioned over or under at least one flexible element.

In some embodiments, when the flexible elements of a single segment are expanded to a fully expanded open state, and rotated they form a circular shape, for example shape 532 that has a diameter, for example diameter 527 of 5-12 mm. In some embodiments, flexible elements of segments located at the proximal part of the electrode array are expanded to a larger diameter than flexible elements of distal segments, for example segment 528. Alternatively, flexible elements of segments located at the proximal part of the electrode array are opened to a smaller diameter than flexible elements of distal segments. Optionally, the flexible elements of all segments of the electrode array are expanded to a similar diameter. In some embodiments, the flexible elements of each segment are expanded to a different diameter compared to the flexible elements of other segments.

In some embodiments, each of the expanded flexible elements is configured to expand independently from the rest of all the other flexible elements. In some embodiments, independent expansion of each of the flexible elements allows, for example to adjust the expansion of at least part of the electrode array according to diameter variations along the blood vessel, for example along the CS.

In some embodiments, the flexible elements of the first two distal segments of electrode array 500, segments 520 and 538 expand to a diameter of 6-9 mm, for example 7 or 8 mm. In some embodiments, the flexible elements of the three proximal segments of electrode array 500, segments 522, 534, and 536 expand to a diameter of 9-13 mm, for example 11 or 12 mm.

In some embodiments, expansion of at least one flexible element of the electrode array allows to anchor the electrode array within a blood vessel, for example the CS and/or to push an electrode connected to the flexible element against the blood vessel inner tissue.

According to some embodiments, each flexible element of the electrode array carries between 1-4 electrodes, for example 2 electrodes. In some embodiments, each electrode array comprises between 2-40 electrodes, for example 30 electrodes.

According to some exemplary embodiments, cylindrical sheath 501 is retracted to allow the deployment of the most proximal electrode or electrodes set. In some embodiments, when electrode array 500 flexible elements are expanded to an open conformation, the distance 540 between the most proximal electrode to the most distal electrode is in the range of 30-90 mm, 30-60 mm, or 50-90 mm for example 64 mm. In some embodiments, the distance 542 between deployed electrodes of one segment to deployed electrodes of the following segment is in the range of 10-50 mm, 25-50 mm or 15-30 mm, for example 16 mm. In some embodiments, when the distal segments 520 and 538 are in an open position, their deployed electrodes fit blood vessels with a diameter of 3-10 mm, or 4-9 mm for example 7 mm or 8 mm. In some embodiments, when the proximal segments, for example segments 522, 534 and 536 are in an open position, their deployed electrodes fit blood vessels with a diameter of 6-12 mm, 6-10 mm, 10-20 mm, for example 10 or 11 mm. In some embodiments, the electrode array is configured to be inserted into tubular blood vessels with a diameter-length ratio of at least 1:3.

According to some exemplary embodiments, an electrode array comprises at least 3 flexible elements, for example flexible arm 526 connected to an elongated shaft, for example shaft 531 in at least three axially spaced-apart locations. In some embodiments, at least one flexible arm is configured to move into an open and relaxed state to allow contact between the flexible element and a blood vessel inner tissue.

Alternatively, at least one flexible arm has an electrode at the distal end of the flexible arm, so when the flexible arm moves into an open and relaxed state, the flexible arm pushes the electrode against the blood vessel inner wall. In some embodiments, during the insertion of the electrode array into the blood vessel, for example the CS, the elongated shaft bends at multiple locations to allow the insertion of the electrode array through the CS ostium. Alternatively, the elongated shaft bends in a position located proximally to the most proximal flexible element, for example position 533. In some embodiments, when the flexible elements are allowed to move into an open and relaxed state, the elongated shaft remains at a straight position when only the flexible elements are allowed to move. In some embodiments, when the flexible elements are at a relaxed state, they are separated from each other, as shown in FIG. 5D, in a way that allows normal blood flow through the electrode array.

Reference is now made to FIG. 5E depicting an electrode array, for example a catheter within a conical-shaped region of a blood vessel, according to some exemplary embodiments of the invention.

According to some exemplary embodiments, catheter 500 is configured to be inserted into the conical region of a blood vessel 542. In some embodiments, flexible arm 526 of the proximal segment 522 is opened to a larger angle degree compared to the opening angle degree of flexible arm 528 of the most distal segment 520. In some embodiments, the difference between the opening degree of distal segment 520 and the proximal segment 522 of catheter 500, allows the catheter to fit into blood vessel regions that have a narrow distal end and a wide proximal end, as shown for example in FIG. 5E.

Reference is now made to FIG. 5F depicting an electrode array, for example the distal end of a catheter, where each electrode is positioned at a different location, according to some embodiments of the invention.

According to some exemplary embodiments, electrode array 500 comprises a tubular body 552, and at least two flexible arms, for example flexible arms 556 and 560, connected to the tubular body in two distinct locations. In some embodiments, each flexible arm, for example flexible arm 556, comprises at least one electrode 558 at its distal end. In some embodiments, each electrode is a ring electrode or a point electrode. In some embodiments, each electrode is a unipolar electrode.

According to some exemplary embodiments, electrode array 500 further comprises a cylindrical sleeve 554 which covers the electrodes of electrode array 500 during the navigation process. In some embodiments, when cylindrical sleeve 554 is retracted in direction 566, the flexible arms, for example flexible arm 556, self-expands to a pre-determined angle with tubular body 552. In some embodiments, when the flexible arm expands to a pre-determined angle, it pushes the electrode at its distal end against the blood vessel tissue. In some embodiments, the predetermined angle of flexible arms located at the proximal part of electrode array 550 is larger than the pre-determined angle of flexible arms located at the distal part of catheter 550, for example flexible arm 560.

In some embodiments, when cylindrical sleeve 554 is retracted in direction 566, after the entire tubular body was confined within cylindrical sleeve 554, the first flexible arm to expand is the most distal flexible arm, for example distal flexible arm 560. In some embodiments, when flexible arm 560 expands, it deploys electrode 562.

In some embodiments, when tubular sleeve 554 is moved in direction 568, after the entire flexible arms expanded, the first flexible arm to collapse is the most proximal flexible arm. In some embodiments, cylindrical sleeve 554 comprises at least one electrode 570 connected to its outer surface. In some embodiments, tubular body 552 comprises at least one electrode 564 at its distal tip. In some embodiments, the at least one electrode 570 and/or the at least one electrode 564 serve as reference electrodes to at least one electrode of electrode array 550.

Reference is now made to FIG. 5G, depicting an electrode array with a plurality of multi/unipolar electrodes, according to some embodiments of the invention.

According to some exemplary embodiments, an electrode array comprises at least one electrode segment. Additionally or optionally, the electrode array comprises at least one electrode at the distal tip of the electrode array. In some embodiments, a catheter device comprising the electrode array includes at least one reference electrode proximally to the electrode array. Optionally the reference electrode is positioned outside the CS, for example in the heart or in the vena cava.

According to some exemplary embodiments, the electrode array, for example electrode array 572 comprises at least 1 electrode segment for example 2, 3, 4, 5, 6, 7 electrode segments, for example electrode segment 574. In some embodiments, each of the electrode segments comprises at least 3 flexible arms, for example flexible arm 576. In some embodiments, each of the flexible arms comprises at least one electrode 578 at the distal section 580 of the flexible arm.

According to some exemplary embodiments, each of the flexible arms is mechanically separated and electrically isolated from all the other flexible arms of the electrode array. In some embodiments, having mechanically isolated flexible arms, allows for example expansion of each flexible arm to a different distance from the electrode array body 571 compared to other flexible arms. Additionally, having mechanically independent flexible arms allows for example adjusting the diameter of the electrode array to fit into blood vessel with varying inner diameters along the blood vessel, for example as in the CS. In some embodiments, expansion of at least one flexible arm allows for example, to place at least one electrode positioned on the flexible arm in contact with the blood vessel inner wall, for example the CS inner wall. Alternatively or additionally, expansion of at least one flexible arm allows, for example anchoring of the electrode array within the blood vessel, for example within the CS.

Reference is now made to FIG. 5H depicting a catheter device comprising an electrode array and at least one reference electrode, according to some embodiments of the invention.

According to some exemplary embodiments, catheter device 590 comprises a steerable catheter body 593 and an electrode array, for example electrode array 572 at the distal section 591 of the catheter body 593. In some embodiments, the catheter device 590 comprises at least one reference electrode, for example reference electrode 592 positioned proximally to the electrode array 572. In some embodiments, the electrode array 572 is configured to be anchored within non-linear blood vessels having variable inner diameters, for example the CS by mechanically separated flexible elements of the electrode array. In some embodiments, when the electrode array 572 is placed within the CS, the at least one reference electrode 592 is positioned within the superior vena cava or the inferior vena cava. Alternatively, the at least one reference electrode 592 is configured to be placed within the right atrium or within the CS.

In some embodiments, the reference electrode is positioned on catheter device 590 in a distance 595 of at least 10 cm from the electrode array 572, for example 15, 20, 25 30, 35, 40 cm and any intermediate numbers. In some embodiments, catheter device 590 comprises at least two spaced apart reference electrodes. In some embodiments, during a mapping procedure the reference electrode is selected based on the distance from an electrode or electrodes of the electrode array that are used for mapping. In some embodiments, the reference electrode is a ring electrode or a point electrode. Optionally, the at least one reference electrode is connected to a flexible element to allow, for example contact between the at least one reference electrode and the inner wall of the blood vessel. In some embodiments, at least one reference electrode and at least one electrode of the electrode array are selected for a mapping procedure based on a distance from an electrical conduction pathway or from a tissue portion that conducts electricity.

### Exemplary Electrodes Connectivity

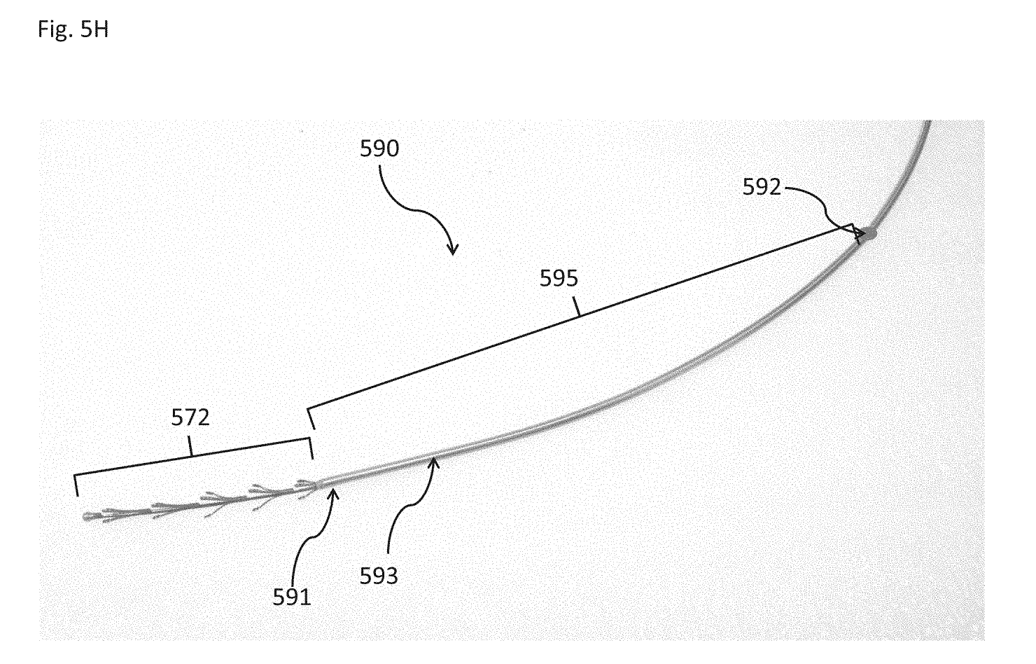

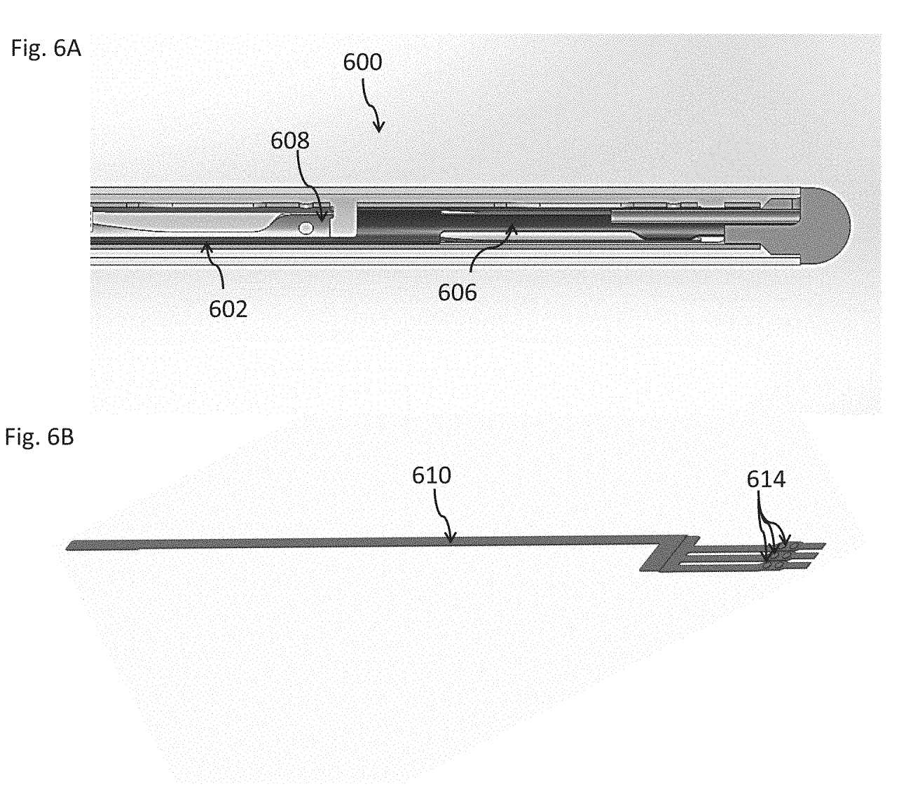

Reference is now made to FIGS. 6A and 6B depicting electrode array components, according to some embodiments of the invention.

According to some exemplary embodiments, as shown in FIG. 6A, an electrode array, for example electrode array 600 comprises an elongated connecting member 602 placed within the central channel of electrode array 600. In some embodiments, the elongated connecting member 602 comprises a wider section 606 at its distal end, which is configured to be attached to a flexible arm, for example flexible arm 608. In some embodiments, flexible arm 608 is made from elastic or super elastic materials, for example Nitinol. In some embodiments, flexible arm 608 comprises at least one hole, to allow fitting at least one electrode. According to some embodiments, elongated member 602 and wider section 606 are printed circuit board (PCB) components, configured to be placed within the central channel of catheter 600.

According to some exemplary embodiments, as shown in FIG. 6B, PCB component, for example PCB component 610, is elongated and comprises at least one side extension 612 at its distal part. Optionally, PCB component 610 comprises a single side extension 612 for every segment along the electrode array. In some embodiments, each side extension 612 comprises at least one spaced-apart elongated extension 613, for example 1, 2, 3, 4, 5, 6 extensions. In some embodiments, elongated extension 613 comprises at least one electrode 614 at its distal end, for example 1, 2, 3, 4 electrodes.

According to some exemplary embodiments, PCB component 610 is positioned within the electrode array body, and each of the side extensions, for example side extension 612 is attached to each segment of the electrode array, for example to the base ring 506 as shown in FIGS. 5A and 5B. Optionally, each PCB component has a single side extension 612 and side extension 612 is attached to a single segment of the electrode array.

In some embodiments, the electric wiring for electrode 614 is embedded within PCB component or connected to its surface.

### Exemplary Catheter Handle

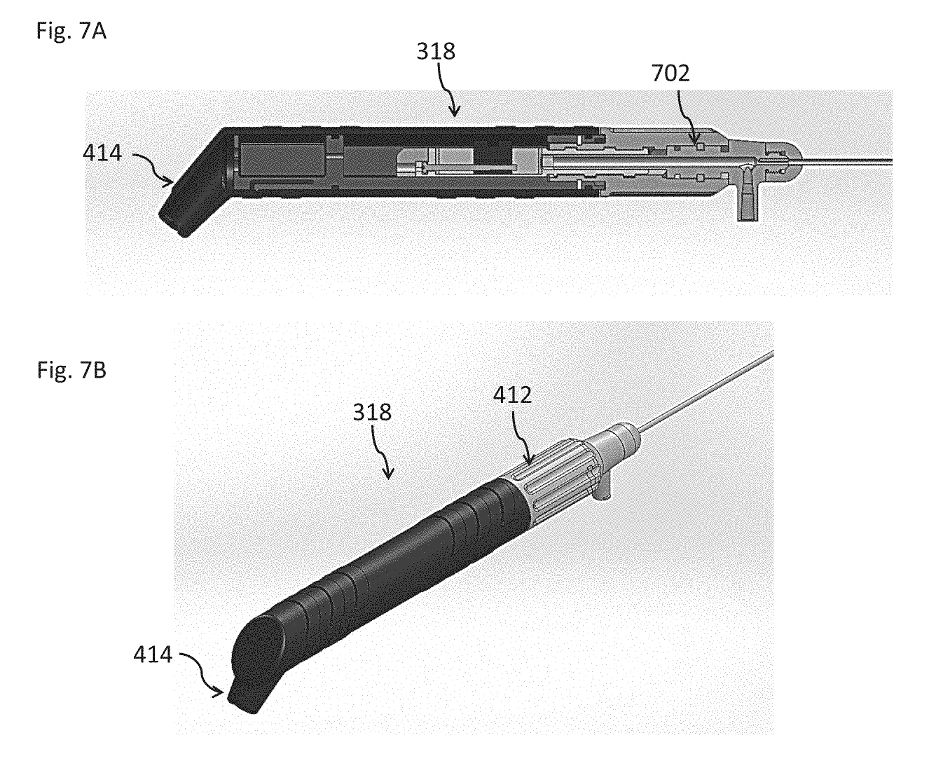

Reference is now made to FIGS. 7A and 7B depicting a catheter handle, according to some embodiments of the invention.

According to some exemplary embodiments, a catheter handle, for example catheter handle 318, controls the navigation of an electrode array into a selected blood vessel, for example the CS. In some embodiments, a catheter handle comprises a housing 700 and a control component, for example control component 412 which by turning into a desired direction, controls electrode array navigation and/or electrodes deployment. In some embodiments, catheter handle 318 further comprises a wiring output, for example wiring output 414 to allow wires traveling from electrodes of an electrode array located at the distal end of the catheter, to pass through the catheter handle. In some embodiments, wiring output 414 allows the passing of electrodes wiring through the catheter handle to an EP measuring device, for example, as shown in FIG. 4.

In some embodiments, catheter handle 318 further comprises a control mechanism 702 which is mechanically connected to a screw-like component 704. In some embodiments, control mechanism threads are connected to screw-like component 704 threads, in a way that rotation of control mechanism 702, for example when control component 412 is turned, allows rotation of screw-like component 704.

In some embodiments, rotation of component 704 allows bending of the electrode array and/or movement of a sheet covering the electrode array electrodes.

Exemplary Electrode Array within the CS

According to some exemplary embodiments, an electrode array with at least two independent expanding elements is inserted into a blood vessel which is located near signal conduction pathways of the heart. In some embodiments, some of the expanding elements allow anchoring of the electrode array within the blood vessel, while other expanding elements carry at least one electrode, which allows EP measurements and/or electric field application and/or delivery of RF energy to the blood vessel tissue. In some embodiments, at least some of the expanding elements allow anchoring of the electrode array within the blood vessel by applying a force against the blood vessel tissue. In some embodiments, by using multiple expanding elements to anchor the electrode array within the CS, it is possible to apply a small force by each of the multiple expanding elements that will be sufficient to anchor the electrode array and will not cause any injury to the blood vessel tissue.

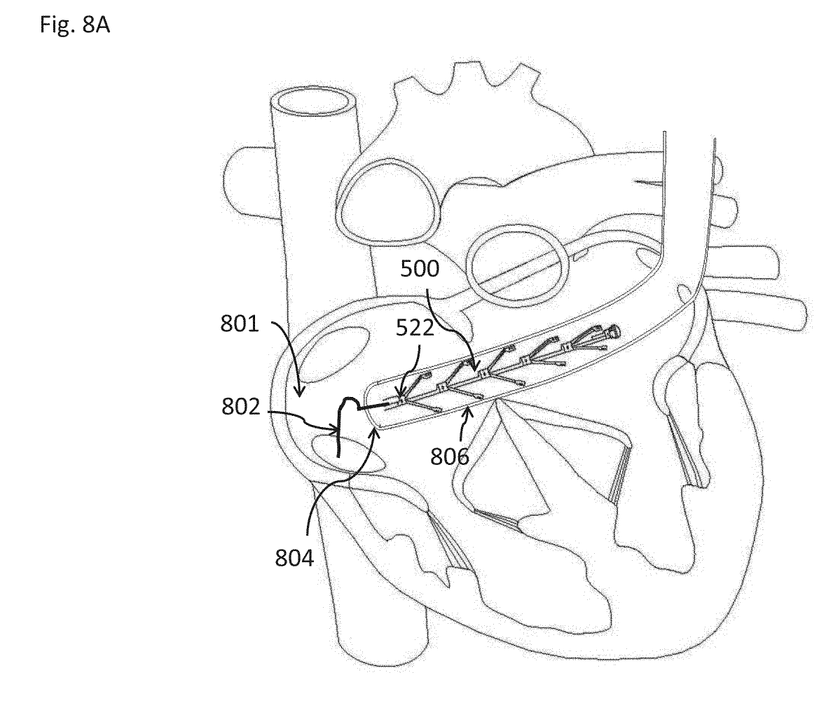

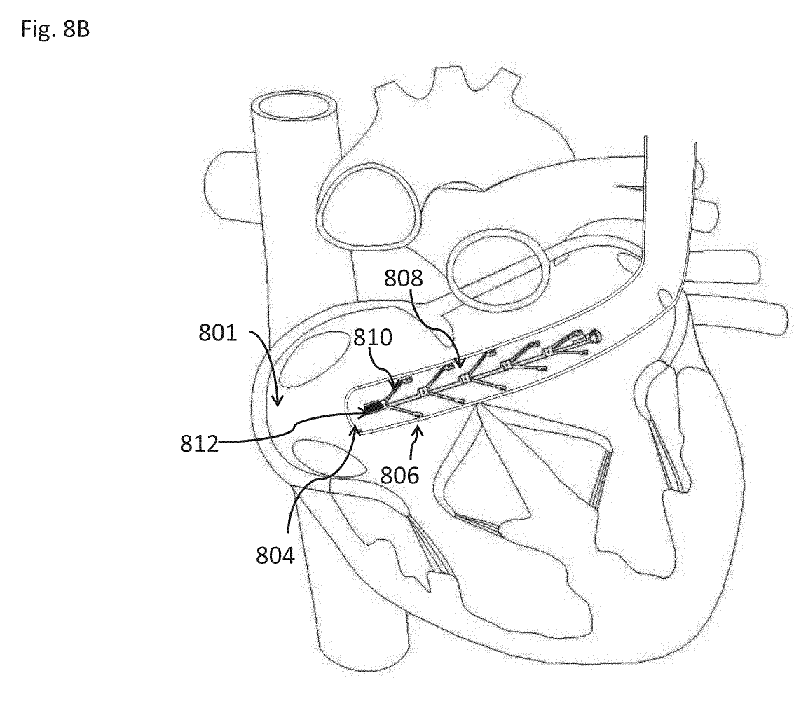

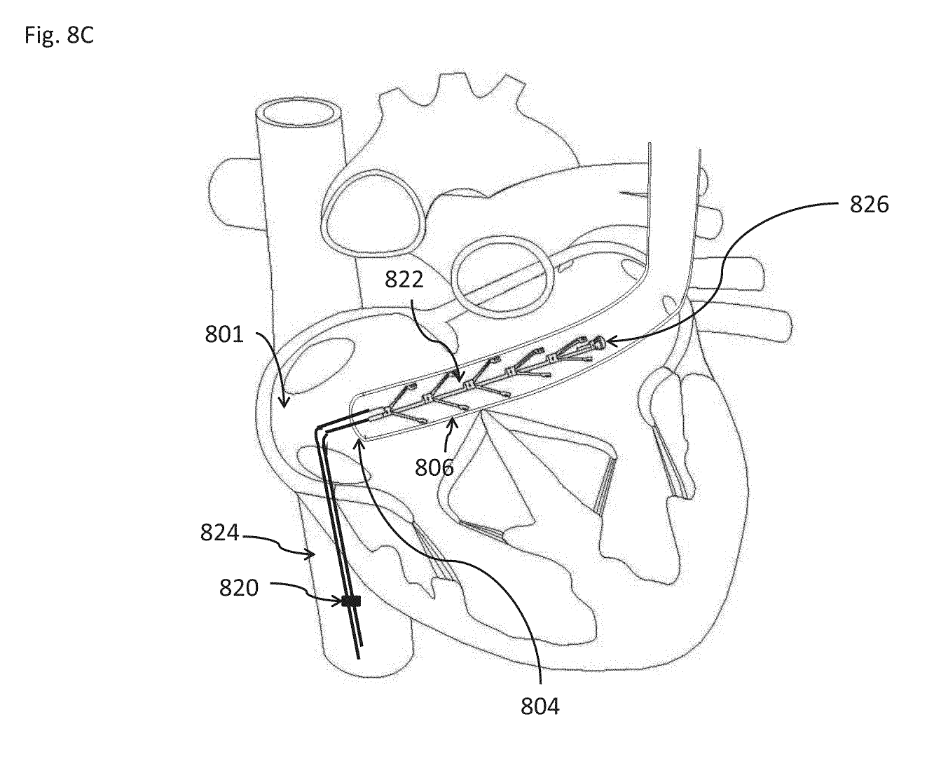

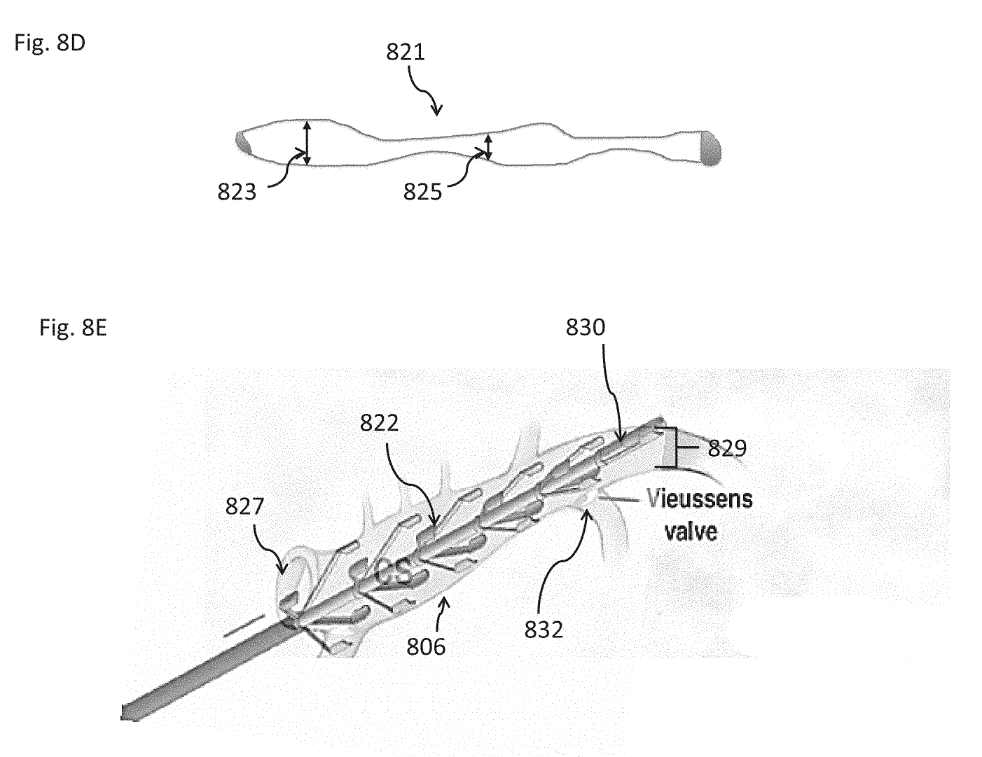

Reference is now made to FIG. 8A depicting an electrode array, for example the distal end of a catheter within the CS of the heart, according to some embodiments of the invention. According to some embodiments, the electrode array allows to deploy at least one selected electrode at a desired location within the CS of an adult.

According to some exemplary embodiments, an electrode array, for example electrode array 500, is navigated using a guide wire, for example guide wire 802 through the right atria 801 and the CS ostium 804, into the CS 806 with the electrode array electrodes in a closed conformation.

In some embodiments, electrode array 500 is configured to fit into conical regions of blood vessels, for example the section of the CS which is closer to the CS ostium. In some embodiments, when the electrode array is placed in a desired location within the CS, for example, when the most proximal segment 522 is placed at the proximal end of the CS, near ostium 804, at least one of the electrodes is deployed. In some embodiments, the electrode is deployed by expansion of a flexible element, for example flexible arm 526, which pushes the electrode against the CS inner tissue. In some embodiments, some flexible elements expand and apply force against the CS inner tissue without pushing an electrode against the tissue. In some embodiments, when electrodes located at the distal part of electrode array 500 are deployed, they make contact with a tissue located at a narrow diameter section of the CS. In some embodiments, when electrodes located at the proximal part of electrode array 500 are deployed, they make contact with a tissue located at a wider diameter section of the CS.

Reference is now made to FIG. 8B depicting an electrode array, which is not connected via wires to an external device. According to some exemplary embodiments, electrode array 808 is inserted into a blood vessel, for example CS 806, through the CS 804. In some embodiments, when electrode array 808 is positioned at a desired location, at least one flexible element out of at least two axially spaced-apart flexible elements, for example flexible arm 810, expands and applies a force against the CS inner tissue. In some embodiments, the applied force allows anchoring of electrode array 808 within CS 806, and is adjusted not to cause any injury to the tissue. In some embodiments, at least one flexible element of electrode array 808 comprises at least one electrode at its distal tip, in a way that expansion of the flexible element pushes the at least one electrode against the CS inner tissue. In some embodiments, each electrode of electrode array 808 is connected via wires to main module 812. According to some embodiments, main module 812 comprises a control circuitry, a power supply component, and a communication circuitry. Optionally, main module 812 comprises a pulse generator for generating electrical pulses. In some embodiments, main module 812 records EP measurements delivered from electrode array 808 electrodes. In some embodiments, main module 812 delivers electric pulses to electrode array 808 electrodes, which are then applied as an electric field to the CS inner tissue.

In some embodiments, the desired force to be applied by at least one deployed electrode and/or flexible element of the electrode array on the blood vessel inner tissue, for example the CS tissue, is sufficient to ensure the contact of the deployed electrode with the tissue. Optionally, the desired force to be applied by at least one deployed electrode and/or flexible element of the electrode array on the blood vessel inner tissue is sufficient to ensure anchoring of the electrode array within the blood vessel. In some embodiments, the desired force is determined based on the tissue strength and/or elasticity and/or thickness. In some embodiments, the desired force to be applied by the at least one deployed electrode is adjusted to prevent an injury, for example tearing of the tissue by the applied force. In some embodiments, the desired force to be applied by the at least one deployed electrode and/or at least one flexible element on the blood vessel tissue is in the range of 0.05-40 gr/contact point, for example 0.1-20 gr/contact point or 2-30 gr/contact point. In some embodiments, the force applied by at least one deployed electrode is measured by a force sensor, for example force sensor 507 located near the at least one electrode. Alternatively, the force sensor is located near the contact point of at least one electrode or at least one flexible element with the blood vessel tissue.

Reference is now made to FIG. 8C depicting an electrode array placed within the CS, with at least one reference electrode positioned within the vena cava, according to some embodiments of the invention.

According to some exemplary embodiments, the electrode array for example electrode array 822 is placed at least partly within the CS. In some embodiments, the electrode array comprises at least one reference electrode, for example reference electrode 820 configured to be positioned outside of the CS. Optionally, the at least one reference electrode is positioned within the vena cava 824.

In some embodiments, the distance between the reference electrode and the tip 826 of the electrode array is at least 20 cm, for example 25, 30, 35, 40, 45 cm and any intermediate numbers. In some embodiments, the reference electrode is a ring electrode, optionally configured to make contact with the vena cava wall. In some embodiments, the reference electrode, for example reference electrode 820 is used for unipolar mapping, optionally to reduce noise during the mapping procedure. In some embodiments, at least two reference electrodes are positioned within the vena cava. Optionally, the reference electrode is selected based on the position of at least one electrode within the CS, and/or based on the distance between the reference electrode and the at least one electrode within the CS.

Reference is now made to FIGS. 8D and 8E, depicting an electrode array positioned within non-linear tubular structures having varying inner diameters, for example the CS, according to some embodiments of the invention.

According to some exemplary embodiments, the electrode array comprises self-expandable elements, which are mechanically independent to allow, for example expansion of each self-expandable element to a different distance and/or to a different diameter from other self-expandable elements. In some embodiments, independent expansion of the self-expandable elements allows, for example adaption of the electrode array within tubular blood vessels with varying inner diameter, for example blood vessel 821. In some embodiments, blood vessel 821 has a variable inner diameter, for example diameter 823 and diameter 825 along the blood vessel, where one diameter is larger than the other. In some embodiments, independent expansion of the self-expandable elements, allows for example contact between some electrodes positioned on the self-expandable elements and the blood vessel inner wall along the blood vessel. In some embodiments, the range over which self-expandable elements apply an allowed and/or a sufficient radial force on the blood vessel wall upon expansion is determined based on the diameter variations of the blood vessel. Optionally, an electrode array or a catheter device is selected based on the diameter variations of the blood vessel.

According to some exemplary embodiments, the electrode array, for example electrode array 822 is placed within the CS 806. In some embodiments, the electrode distal section, for example distal section 830 is positioned proximally to the vieussens valve 832 or proximally to the connection between the CS and the great cardiac vein. In some embodiments, the diameter of CS 806 in the orifice region 827 is larger than the diameter 829 of the CS 806 near the connection to the cardiac vein. In some embodiments, the self-expandable elements of the electrode array 822 expand independently, and push the electrodes against the inner wall of CS 806 at sections of CS 806 having different diameter sizes as previously described.

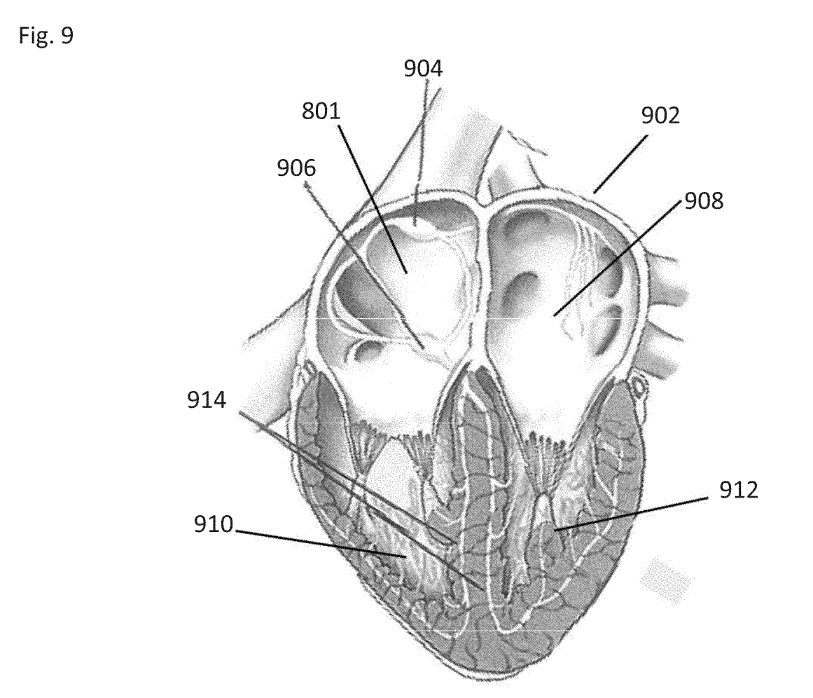

Reference is now made to FIG. 9 depicting signal conduction pathways in the heart, according to some embodiments of the invention. According to some embodiments, an electrode array for example electrode array 500 or electrode array 808 is positioned within the blood vessel based on the distance from signal conduction pathways in the heart. In some embodiments, at least one selected electrode is deployed based on its distance from these pathways. In some embodiments, the electrode array is positioned near the coronary ostium, and/or deploys at least one electrode at this position to allow EP measurements of signals travelling from the right atria to the ventricles, optionally through the Atrioventricular (AV) node.

According to some embodiments, signals conduction pathways of the heart conduct signals from the right atria 801, to the left atria 908, the right ventricle 910, and the left ventricle 912.

According to some exemplary embodiments, the CS of heart 902 is connected to right atria 801 through the CS ostium. In some embodiments, the CS is positioned in a relatively close distance to signal conduction pathways of the heart which originate from the sinoatrial node (SA node) 904, located at the upper region of right atria 801. In some embodiments, some of the SA node 904 signals are delivered to AV node 906 directly or through AV node extensions, which are located in close proximity to the CS. In some embodiments, from the AV node, some of the signals are delivered to the heart ventricles through the bundles of His 914, and Purkinje fibers.

According to some exemplary embodiments, placing an electrode array, for example electrode array 500 at least partly within the CS, allows to measure electrical parameters associated with the signals propagation through the heart tissue, for example between SA node 904 and AV node 906. Optionally, placing an electrode array, for example electrode array 500 at least partly within the CS allows to apply an electric field to the heart tissue, for example to AV node 906 and/or AV node extensions.

### Exemplary Helical Electrode Arrays

According to some exemplary embodiments, an electrode array has a helical main body with electrodes connected to its surface.

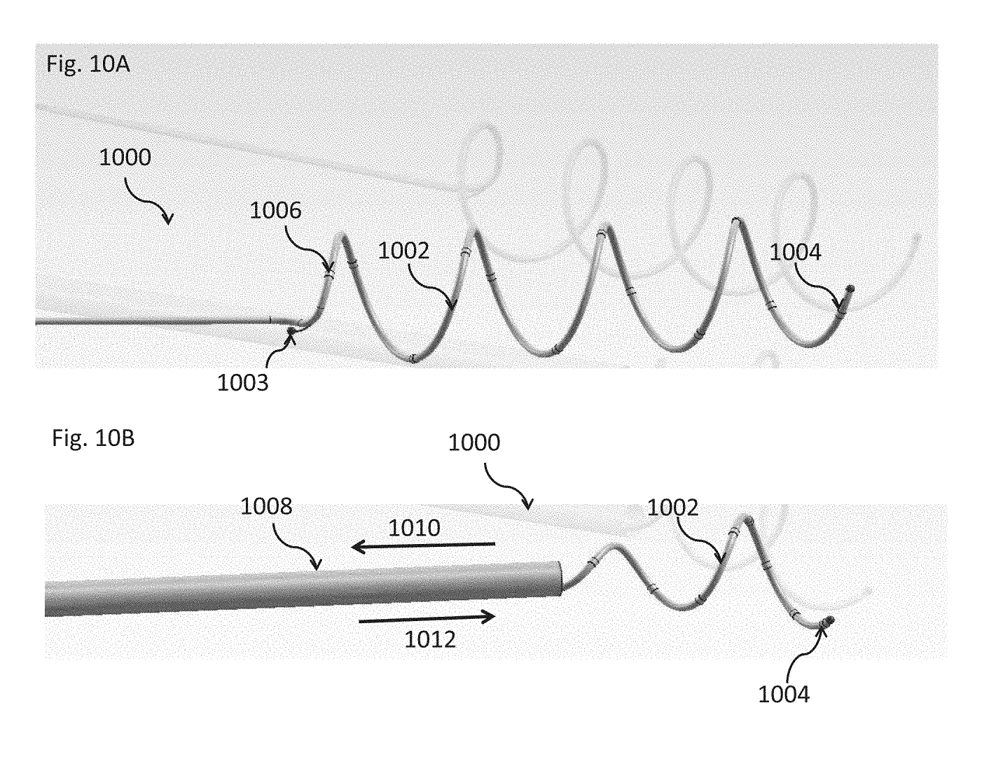

Reference is now made to FIGS. 10A and 10B depicting a helical electrode array with electrodes on its outer surface, according to some embodiments of the invention. According to some exemplary embodiments, an electrode array, for example electrode array 1000 comprises a self-expandable tubular body 1002 and electrodes connected to the tube in predetermined locations. In some embodiments, when self-expandable tubular body 1002 is expanded into an open conformation, it acquires an open helical shape with a diameter of 5-12 mm, 7-10 mm, to allow fitting the electrode array within a blood vessel, for example the CS. In some embodiments, the length of the electrode array section that is placed within the blood vessel is 10-120 mm, 20-100 mm or 50-120 mm. In some embodiments, the open helical shape structure, pushes at least one electrode, for example electrodes 1004 and 1006 against the blood vessel inner wall, to allow contact between the at least one electrode and the blood vessel tissue. In some embodiments, when tubular body 1002 expands to an open conformation, it anchors electrode array 1000 within the blood vessel. In some embodiments, as shown in FIG. 10A tubular body expands to an open helical shape with a tubular structure that has a similar diameter along the electrode array body.

According to some exemplary embodiments, during the navigation process self-expandable tubular body 1002 is confined within cylindrical sheath 1008 of the helical electrode array, for example electrode array 1000. In some embodiments, when electrode array 1000 is positioned in a desired location within a blood vessel, for example the CS, cylindrical sheath 1008 is retracted in direction 1010 to expose at least one selected electrode. In some embodiments, when cylindrical sheath 1008 is retracted, the at least one exposed electrode is allowed to make contact with the blood vessel tissue. In some embodiments, retraction of cylindrical sheath 1008 allows self-expandable tubular body 1002 to acquire an open helical conformation, as shown in FIG. 10A, and to push the at least one exposed electrode against the blood vessel tissue. In some embodiments, when tubular body 1008 is retracted in direction 1010, the first electrode to be exposed is the most distal electrode, for example electrode 1004.

In some embodiments, electrode array 1000 comprises at least one reference electrode, for example electrode 1003 located at the most proximal end of self-expandable tubular body 1002. Alternatively, electrode array 1000 comprises at least one reference electrode connected to the distal tip of the self-expandable tubular body 1002. Optionally, electrode 1003 is deployed outside of the CS.

According to some exemplary embodiments, if additional electrodes need to be deployed, cylindrical sheath is further retracted in direction 1010. In some embodiments, if the electrode array, for example electrode array 1000 needs to be re-positioned or retracted from the blood vessel, then cylindrical sheath is moved in direction 1012 to cover the electrodes. In some embodiments when the electrodes are covered, self-expandable tubular body 1002 is confined within cylindrical sheath 1008, optionally in a closed helical conformation.

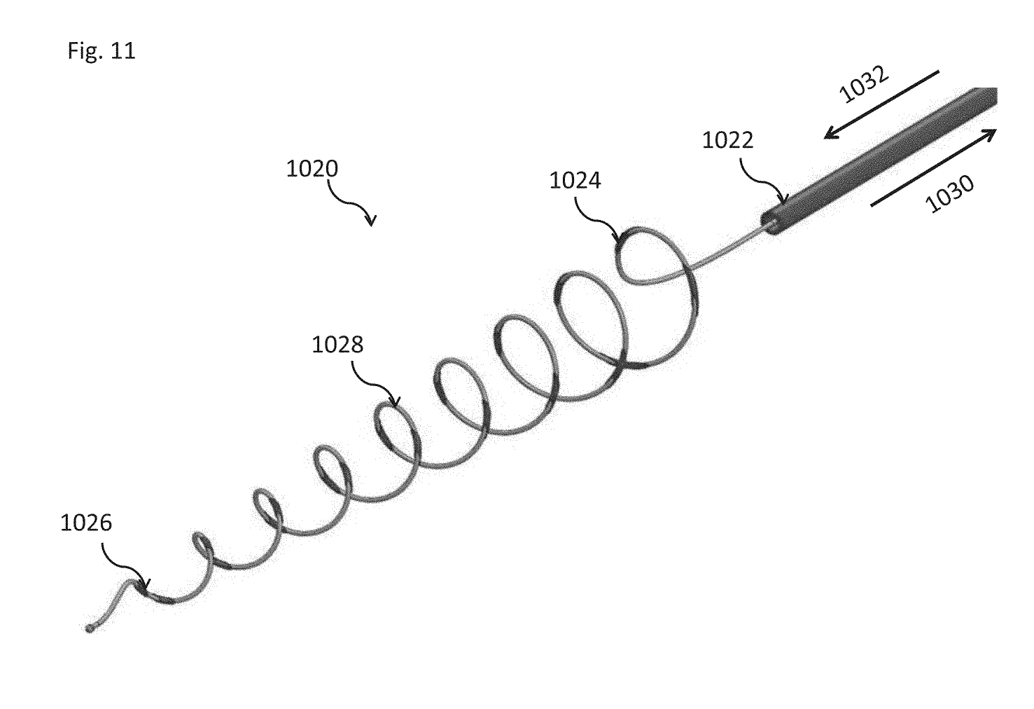

Reference is now made to FIG. 11 depicting a helical electrode array, according to some embodiments of the invention.

According to some exemplary embodiments, a helical electrode array, for example electrode array 1020, comprises a self-expandable tubular body 1028, and at least one electrode, for example electrode 1026 connected to the tubular body. In some embodiments, during the navigation process, the tubular body is confined within a cylindrical sheath, for example cylindrical sheath 1022, in a closed helical conformation. In some embodiments, when cylindrical sheath is retracted, as shown in FIG. 11, self-expandable tubular body 1028 acquires an open conical conformation, which fits blood vessels with conical sections, for example the CS. In some embodiments, the open conical conformation has a diameter of 5-12 mm. In some embodiments, the length of the electrode array section that is placed within the blood vessel is 10-120 mm, 20-100 mm or 50-120 mm. In some embodiments, when cylindrical sheath 1022 is retracted from a fully closed position in direction 1030, the most distal electrode is deployed first, for example electrode 1026. In some embodiments, when self-expandable tubular body 1028 is in an open conformation, its proximal electrode, for example electrode 1024 is positioned in a wider helical section compared to its distal electrodes, for example electrode 1026 which are found in a narrow helical section of electrode array 1020. In some embodiments, by moving cylindrical sheath 1022 in direction 1032, electrode array 1020 electrodes are collapsed and are confined within the cylindrical sheath, as described in FIG. 10B.

According to some exemplary embodiments, using a self-expandable helical conformation as shown in FIGS. 10A and 10B allows to deploy selected electrodes of the electrode array within a blood vessel at different radial and/or axial positions without using flexible arms, for example flexible arms 526 and 525 of electrode array 500.

According to some exemplary embodiments, some of electrode array 1000 and 1020 elements, for example self-expandable tubular bodies 1028 and 1002 are made from elastic or super-elastic materials, for example Nitinol.

### Exemplary Stent-Like Electrode Arrays

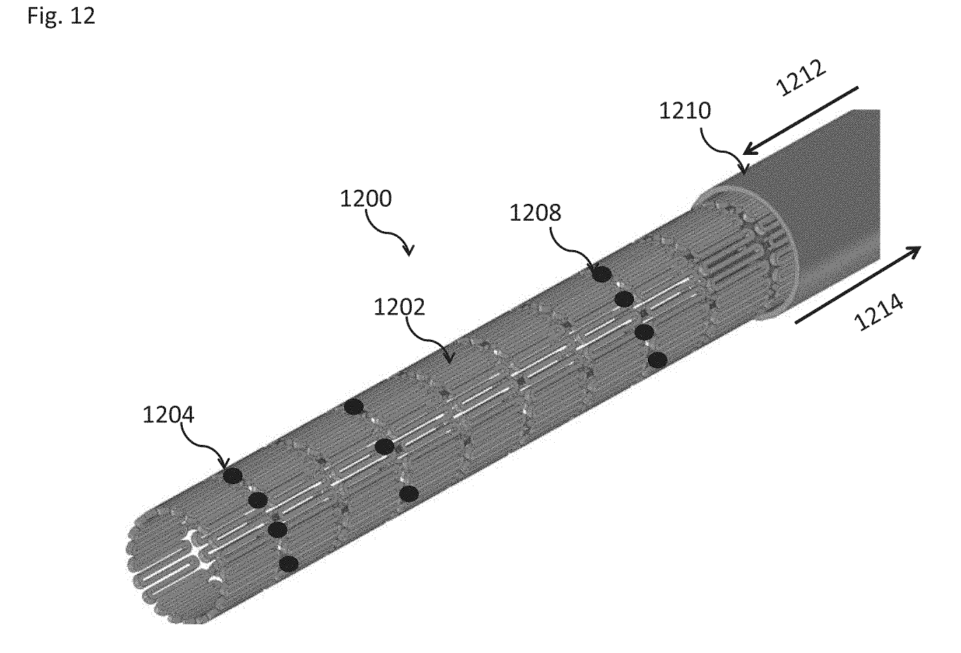

According to some exemplary embodiments, an electrode array, for example electrode array 1200 comprises a stent-like structure with at least one electrode connected to its outer surface. Reference is now made to FIG. 12 depicting a stent-like electrode array, according to some embodiments of the invention.

According to some exemplary embodiments, an electrode array, for example electrode array 1200, comprises a main tubular body 1202, with at least one electrode, for example electrode 1024 connected to its surface. In some embodiments, tubular body 1202 comprises electrodes located in predetermined locations on its surface, for example electrode 1204 located at a distal location, and electrode 1208, which is located at a more proximal location.

In some embodiments, during the navigation process, tubular body 1202 is confined within cylindrical sheath 1210. In some embodiments, when electrode array 1200 reaches a desired location within a blood vessel, for example the CS, cylindrical sheath is retraced in direction 1214 to deploy electrodes connected to tubular body 1202. In some embodiments, the cylindrical sheath is retracted until a selected electrode is deployed.

According to some exemplary embodiments, tubular body 1202 has a fixed diameter tubular structure when it is confined within cylindrical sheath 1210 and when it is exposed, after cylindrical sheath 1210 was retracted. Optionally, when cylindrical sheath 1210 is retracted, tubular body 1202 acquires a conical shape, with a wide distal end and a narrow proximal end.

In some embodiments, the length of the tubular body that is placed within the blood vessel is in the range of 10-120 mm, 20-100 mm or 50-120 mm. In some embodiments, tubular body diameter is in the range of 5-12 mm.

### Exemplary Flexible Arms

According to some exemplary embodiments, an electrode array comprises at least one flexible arm. In some embodiments, the flexible arm is a self-expanding arm, configured to attach at least one electrode connected to the arm, to the blood vessel tissue. In some embodiments, the flexible arm attaches the at least one electrode to the tissue, by pushing it against the blood vessel wall, for example a vein.

In some embodiments, the flexible arm is configured to attach the at least one electrode in a desired direction for EP measurements and/or for electric field application and/or RF energy delivery.

According to some exemplary embodiments, the flexible arm pushes the at least one electrode against the blood vessel wall with a force that allows contact between the at least one electrode and the tissue, but does not cause any injury to the tissue, for example tearing of the blood vessel wall. Optionally, the flexible arm of an electrode array, is pushed against the blood vessel tissue to allow anchoring of the electrode array within the blood vessel.

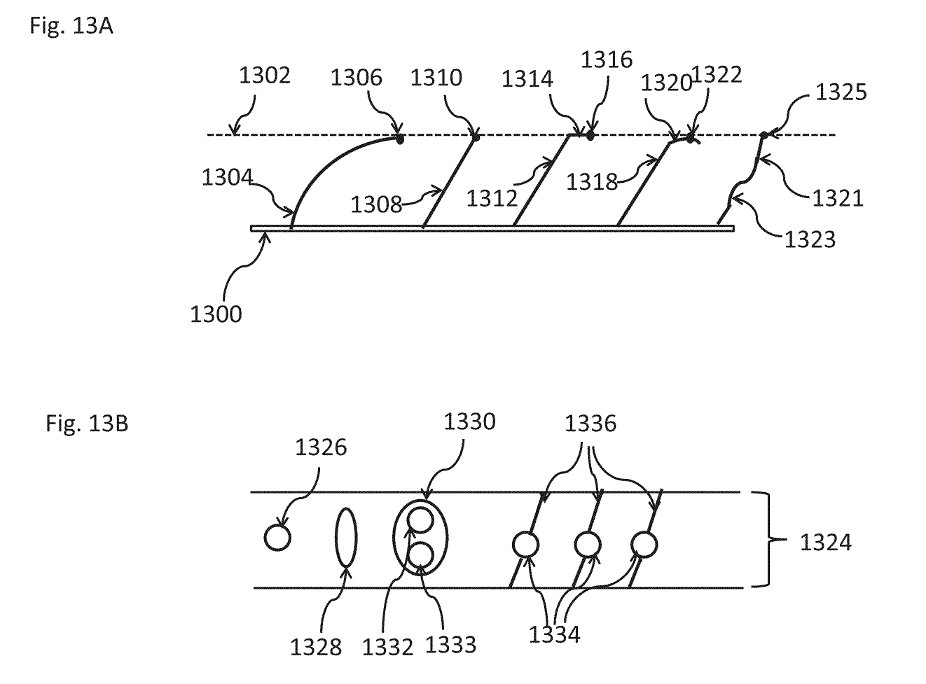

Reference is now made to FIG. 13A, depicting embodiments of flexible arms, according to some embodiments of the invention.

According to some exemplary embodiments, an electrode array comprises at least one flexible arm connected to an elongated body 1300, and carries at least one electrode near its distal end. In some embodiments, the flexible arm is configured to push the at least one electrode against the blood vessel tissue 1302. In some embodiments, the flexible arm, for example arm 1304 is a curved arm, and carries at least one electrode 1306. In some embodiments, electrode 1306 is placed near the distal end of 1304, and faces the blood vessel tissue. Alternatively, the flexible arm, for example flexible arm 1308 is a straight arm, and carries at least one electrode, for example electrode 1310 near its distal end.

In some embodiments, the flexible arm, for example flexible arm 1312 is straight, and has a straight end plate, for example end plate 1314 connected to its distal end. In some embodiments, end plate 1314 comprises at least one electrode, for example electrode 1316 positioned to face the blood vessel tissue. Alternatively, the flexible arm, for example flexible arm 1318 has a curved end plate, for example end plate 1320, connected to its distal end. In some embodiments, curved end plate 1320 comprises at least one electrode, for example electrode 1322, positioned to face the blood vessel tissue.

In some embodiments, the flexible arm, for example, flexible arm 1321 comprises an s-shaped section, for example section 1323 which allows additional flexibility when pushing electrode 1325 against the blood vessel tissue.

### Exemplary Electrode-Tissue Contact Points

Reference is now made to FIG. 13B depicting contact points between an electrode array electrode and a blood vessel inner tissue, according to some embodiments of the invention.

According to some exemplary embodiments, an electrode array pushes at least one electrode with a radial force, to allow contact between the electrode and the inner surface of a blood vessel, for example blood vessel 1324. In some embodiments, the radial force is adjusted not to cause an injury to the tissue. In some embodiments, the contact point between at least one electrode of the electrode array and the tissue is a round contact point 1326, or an oval contact point 1328. In some embodiments, the area of each contact point is at least 1 mm², for example 1 mm², 2 mm², 3 mm². In some embodiments, a contact point, for example contact point 1330 comprises two electrode contacts, for example electrode contacts 1332 and 1333. In some embodiments two electrodes contacts are electrically inter-connected. Alternatively, the two electrodes contacts are electrically isolated from each other.

In some embodiments, electrodes are pushed by a helical self-expandable body, for example tubular bodies 1002 and 1008 against the blood vessel inner wall.

In some embodiments, the helical body, for example helical body 1336 pushes the electrodes to axially spaced-apart contact points 1334 with the blood vessel inner tissue.

### Exemplary Electrical Mapping

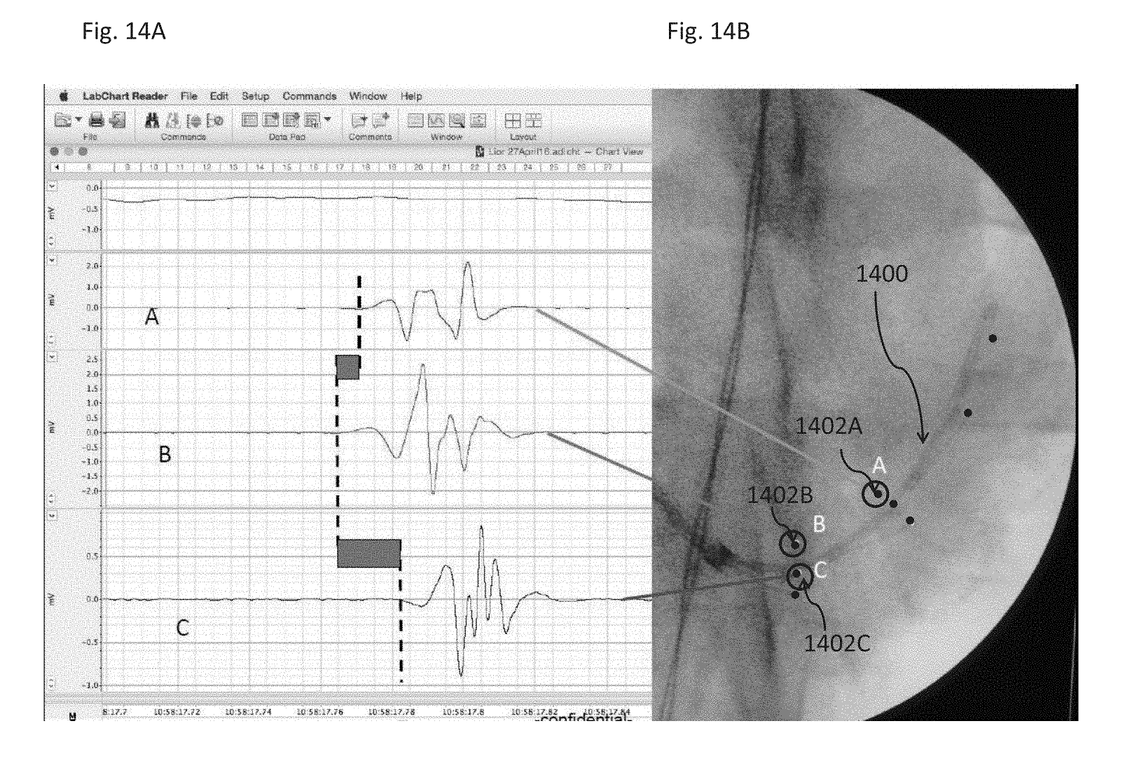

Reference is now made to FIGS. 14A and 14B depicting the results of an electrical mapping procedure from within the CS, according to some embodiments of the invention.

According to some exemplary embodiments, an electrode array with self-expandable elements, for example electrode array 1400 is inserted into the CS of swines. In some embodiments, for example as shown in FIG. 14B, the electrode array is navigated and visualized within the CS using fluoroscopy. In some embodiments, electrode array 1400 is a 9 French electrode array. Optionally, electrode array 1400 comprises 32 electrodes. In some embodiments, each of the electrodes is made from platinum and has a diameter of 0.4 mm. In some embodiments, the distance between adjacent electrodes is 2 mm. In some embodiments, each of the electrodes was constructed on a Nitinol frame with printed ultraflexible wave-shaped platinum-iridium wires.

In some embodiments, unipolar and/or bi-polar mapping can be performed during sinus rhythm, atrial pacing and induced atrial fibrillation, by recording from at least one electrode at position 1402A, 1402B and 1404C respectively. In some embodiments, for example as shown in FIG. 14A graphs A, B and C represents local electrocardiogram (ECG) recordings during sinus rhythm.

According to some exemplary embodiments, positioning of the catheter in the CS can reveal apposition of the electrodes along its axial and coronal dimensions. In some embodiments, activation mapping can reveal significant anisotropic conduction patterns along the CS, with areas of relatively fast axial conduction velocity (2.1-3.2 mm/ms) with corresponding slow coronal conduction velocity (0.6-1.3 mm/ms) at the same longitudinal plane, as can be seen for example in FIGS. 14A and 14B demonstrating that electrical activation on electrode 1402A occurs before the activation on electrode 1402C. Additionally, the activation mapping can reveal a complex non-linear activation sequence along the CS in sinus versus paced rhythms. In some embodiments, the activation mapping can reveal critical zones of conduction during atrial fibrillation, possibly dictating left atrial signal propagation into the AV node.

It is expected that during the life of a patent maturing from this application many relevant electrode arrays will be developed; the scope of the term electrode array is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An electrode array configured to be inserted at least partially into a blood vessel comprising:
a self-expandable array body;
at least two axially spaced-apart electrodes connected to said array body;
wherein at least part of said array body expands to an open helical conformation with a fixed or varying diameter of 5-12 mm, and pushes at least one selected electrode against a blood vessel inner tissue with a force designed not to damage venous tissue.

2. The electrode array of claim 1, wherein said array body comprises:
an elongated shaft;
at least three spaced-apart flexible elements, connected with their proximal ends to said elongated shaft in at least three axially spaced-apart locations along said shaft, wherein at least one of said three flexible elements moves to a relaxed state and pushes said blood vessel inner tissue with its distal end when said array body expands;
wherein at least one of said flexible elements is connected to at least one of said electrodes at its distal end, and wherein said array body expands and acquires said open helical conformation, said flexible element pushes said electrode against said blood vessel inner tissue.

3. The electrode array of claim 1, wherein expansion of said array body into said open helical conformation, anchors said electrode array at least partly within a tubular blood vessel with a diameter-length ratio of at least 1:3.

4. The electrode array of claim 1, wherein said electrode is pushed against the blood vessel inner tissue with a force of 2-30 gr when said array body expands into said open helical conformation.

5. The electrode array of claim 2, wherein said flexible elements make contact with said blood vessel inner tissue with a force of 2-30 gr when said array body expands.

6. The electrode array of claim 1, wherein said electrodes contact point with said blood vessel inner tissue allows EP measurements and/or electric field application when said electrode is pushed against the tissue.

7. The electrode array of claim 1, wherein said electrodes are pushed against the inner tissue of the coronary sinus with a force that does not cause an injury of the tissue.

8. The electrode array of claim 1, wherein said open helical conformation of said array body is a helical conformation with a fixed diameter in the range of 5-12 mm.

9. The electrode array of claim 1, wherein said open helical conformation of said array body is a helical conformation with a varying diameter of 5-12 mm.

10. The electrode array of claim 1, wherein said open helical conformation of said array body is a helical conformation with a conical shape that has a smaller diameter at its distal section and a wider diameter at its proximal section.

11. The electrode array of claim 10, wherein said smaller diameter is in the range of 5-8 mm, and wherein said wider diameter is in the range of 9-12 mm.

12-13. (canceled)

14. The electrode array of claim 2, wherein each of said flexible elements found in said relaxed state, form a circle with a diameter of 6-12 mm when said electrode array rotates.

15. The electrode array of claim 2, wherein flexible elements connected to said elongated shaft at proximal locations, form a circle with a diameter of 10-12 mm when they are found in said relaxed state, and
wherein flexible elements connected to said elongated shaft at distal locations, form a circle with a diameter of 6-8 mm when they are found in said relaxed state and upon rotation of said electrode array.

16. The electrode array of claim 1, further comprising a cylindrical sleeve for covering said electrode array when said electrode array is introduced into said blood vessel and/or for protecting said blood vessel tissue during the insertion of said electrode array into the lumen of said blood vessel.

17. The electrode array of claim 16, wherein movement and/or rotation of said cylindrical sleeve allows expansion or collapse of at least part of said array body.

18. The electrode array of claim 16, wherein movement and/or rotation of said cylindrical sleeve allows deployment and/or collapse of at least one selected electrode.

19. The electrode array of claim 16, wherein movement and/or rotation of said cylindrical sleeve allows deployment and/or collapse of at least one selected flexible element.

20. The electrode array of claim 1, wherein pushing of said at least one electrode and/or at least one flexible element allows anchoring of said electrode array within said blood vessel.

21. The electrode array of claim 1, wherein the distance between two axially spaced apart electrodes, located on two axially spaced apart flexible elements is at least 1 mm.

22-24. (canceled)

25. The electrode array of claim 2, wherein at least one electrode is configured to be pushed by said at least one flexible element to a tissue near the coronary sinus ostium.

26. The electrode array of claim 1, further comprising at least one force sensor located on the array body for measuring the force applied by said array on said blood vessel tissue when said array body expands and/or during the insertion of said electrode array into the lumen of said blood vessel.

27. (canceled)

28. The electrode array of claim 1, further comprising at least one force sensor located near said at least one electrode, configured to measure the force applied by said electrode on said blood vessel tissue when said array body expands.

29. The electrode array of claim 1, further comprising at least one position sensor connected to said array body, configured to measure spatial location and/or orientation of said electrode array within said blood vessel.

30. The electrode array of claim 6, wherein said contact point has an area of 1 mm²-3 mm².

31. The electrode array of claim 1, wherein expansion of said array body contacts less than 30% of said blood vessel inner tissue.

32-39. (canceled)

40. The electrode array of claim 1, wherein at least part of said array body comprises a hollow channel.

41. The electrode array of claim 40, comprising a movable stylet passing through said hollow channel, wherein said stylet is configured to change a conformation of at least part of said hollow array body.

42. The electrode array of claim 1, wherein said electrode array is suitable for navigation using a guide wire.
